(19) United States
(12) Patent Application Publication
(10) Pub. No.: US 2023/0002368 A1
(43) Pub. Date: Jan. 5, 2023

(54) USE OF COMPOUNDS FOR THE PREVENTION AND/OR TREATMENT OF ANKYLOSING SPONDYLITIS, AND CORRESPONDING COMPOSITIONS

(71) Applicant: NUVAMID SA, Epalinges (CH)

(72) Inventors: Guillaume BERMOND, MARSEILLE (FR); Laurent GARCON, SAUSSET LES PINS (FR)

(73) Assignee: NUVAMID SA, Epalinges (CH)

(21) Appl. No.: 17/775,615

(22) PCT Filed: Nov. 27, 2020
(86) PCT No.: PCT/EP2020/083782

§ 371 Date: May 10, 2022

(30) Foreign Application Priority Data
Nov. 28, 2019 (FR) FR1913423

(51) Int. Cl. C07D 421/14; C07D 409/14; C07D 405/14; C07D 401/14; C07D 401/10; A61K 9/00; A61P 19/02

(52) U.S. Cl. CPC C07D 421/14; C07D 409/14; C07D 405/14; C07D 401/14; C07D 401/10; A61K 9/0019; A61K 9/0073; A61K 9/0051; A61P 19/02

(57) ABSTRACT

The invention concerns a compound of formula (I) and/or a compound of formula (Ia) for use in the prevention and/or treatment of ankylosing spondylitis, as well as compositions and combination preparations comprising them.

### FIELD OF THE INVENTION

This invention concerns the use of compounds of formula (I) or (Ia), as well as compositions comprising them, for the treatment and/or prevention of ankylosing spondylitis.

### PRIOR ART

A joint is an assembly, in particular, of two cartilage-covered ends of bones, as well as a synovial membrane enveloping this assembly. The role of the synovial membrane is to facilitate joint movements by secreting a lubricant: synovial fluid. Inflammatory rheumatism consists, in particular, of inflammation of the synovial membrane. An excessive amount of synovial fluid is then secreted, and the synovial membrane thickens abnormally. The soft tissues and osseous surfaces of the joint are then damaged. The joint becomes abnormally swollen and painful, hindering movement.

Ankylosing spondylitis is an inflammatory rheumatic disorder most commonly affecting the spinal column, pelvis, and sacrum, which describe a form of the disease known as the ‘axial’ form. The ‘peripheral’ form affects joints other than the spinal column. Ankylosing spondylitis may also affect the insertion points of the tendons and muscles at the bones, in particular the Achilles tendon, as well as other organs such as the eye, the heart, and the intestines. Ankylosing spondylitis is strongly associated with the presence of the HLA B27 marker. Ankylosing spondylitis is not a rheumatoid arthritis; ankylosing spondylitis primarily occurs in young patients, whilst rheumatoid arthritis and chronic rheumatic disorders primarily affect older patients. The treatments are also different. Moreover, ankylosing spondylitis stiffens and deforms joints, in particular the spinal column. Patients feel stiffening of the spine and joints, and a certain period of ‘derusting’ of the body is necessary in the morning in order for them to be able to move again. Inflammation of the spinal column may develop into ankylosis, as is the case with the sacroiliac joint. Ankylosis is an ossification of the joint damage that ‘welds’ together the various bony parts forming the joints. This ankylosis causes significant loss of mobility and functional impairment. One of the risks for patients is that the ankylosis subsequent to bone formation develops into an abnormal posture of the spine. Currently, there is no curative treatment, and the pathophysiological mechanisms remain unknown.

Ankylosing spondylitis is a disease that develops by means of inflammatory eruptions during which the inflammation is particularly severe, spaced apart by ‘remission’ periods in which the patient can lead a normal life.

Symptomatic treatment of spondylitis primarily comprises administering analgesics to reduce pain, NSAIDs (non-steroidal anti-inflammatory drugs), as well as cortisone or derivatives thereof to reduce inflammation. However, the chronic administration of these drugs causes damage, inter alia, to the stomach, liver, and kidneys. Additionally, the chronic use of cortisone derivatives causes, inter alia, bone fragility, neuropsychiatric effects, muscle wasting, and reduced immunity, leaving the patient vulnerable to infections. Moreover, their efficacy decreases over time, requiring increases in dosage or the use of more aggressive drugs, such as anti-TNF agents, methotrexate, or sulphasalazine, that often cause greater side-effects.

Methotrexate is an anti-cancer agent used to prevent and reduce the number of inflammatory eruptions. However, this drug has numerous side-effects, such as fever, anaemia, respiratory distress, risks of teratogenicity, and bone marrow toxicity, amongst other risks. As such, it is not well tolerated by all patients. Other drugs are used in lieu of or in conjunction with methotrexate, e.g. inhibitors of TNF (Tumour Necrosis Factor), a protein implicated in inflammatory processes. Sulphasalazine, an anti-inflammatory, is also used in the treatment of certain forms of ankylosing spondylitis when the administration of NSAIDs no longer suffices. However, side-effects are common, and include stomach ache, skin rash, and buccal lesions. Moreover, methotrexate and sulphasalazine are not effective against the axial forms of ankylosing spondylitis; their efficacy is limited to the peripheral form. Lastly, in the more severe cases, only surgery can correct the skeletal deformations and disabling forms of the disease.

None of these drugs is free of side-effects. In particular, there is a risk of weakening the immune system, as well as substantial toxicity to the patient's vital organs, such as the liver and kidneys.

Thus, there is a need to develop novel compounds and compositions for the treatment and/or prevention of ankylosing spondylitis (AS) that reduce the disadvantages of the prior art.

### SUMMARY OF THE INVENTION

These objectives are met by the invention as described infra.

This invention concerns a compound of formula (I):

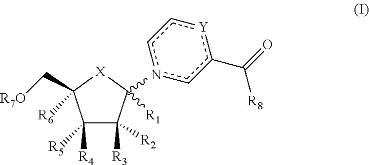

or a pharmaceutically acceptable stereoisomer, salt, hydrate, solvate, or crystal thereof, wherein
X is selected from O, CH₂, S, Se, CHF, CF₂, C═CH₂;
R₁ is selected from H, azido, cyano, C₁-C8 alkyl, C₁-C8 thioalkyl, C₁-C8 heteroalkyl, and OR; wherein R is selected from H and C₁-C8 alkyl;
R₂, R₃, R₄, and R₅ are selected independently of one another from H, halogen, azido, cyano, hydroxyl, C₁-C12 alkyl, C₁-C12 thioalkyl, C₁-C12 heteroalkyl, C₁-C12 haloalkyl, and OR; wherein R is selected from H, C₁-C₁₂ alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)NH(C₁-C₁₂) alkylaryl, C(O)O(C₁-C₁₂) alkylaryl, and C(O)CHR_{AA}NH₂; wherein R_{AA} is a side chain selected from the proteinogenic amino acids;
R₆ is selected from H, azido, cyano, C₁-C8alkyl, C₁-C8 thioalkyl, C₁-C8 heteroalkyl, and OR; wherein R is selected from H and C₁-C8 alkyl;
R₇ is selected from H, P(O)R₉R₁₀, and P(S)R₉R₁₀; wherein
R₉ and R₁₀ are selected independently of one another from OH, OR₁₁, NHR₁₃, NR₁₃R₁₄, C₁-C₆ alkyl, C₂-C₈ alkenyl, C₂-C₈ alkynyl, C₃-C10 cycloalkyl, C₅-C12 aryl. (C₁-C₈) alkylaryl, (C₁-C₈) arylalkyl, (C₁-C₈) heteroalkyl, (C₁-C₈) heterocycloalkyl, heteroaryl, and NHCHR_{A}R_{A′}C(O)R₁₂; wherein:
R₁₁ is selected from a C₁-C10 alkyl, C₃-C10 cycloalkyl, C₅-C18 aryl, C₁-C10 alkylaryl, substituted C₅-C12 aryl, C₁-C10 heteroalkyl, C₃-C10 heterocycloalkyl, C₁-C₁₀ haloalkyl, heteroaryl, —(CH₂)ₙC(O)(C₁-C₁₅) alkyl, —(CH₂)ₙOC(O)(C₁-C₁₅) alkyl, —(CH₂)ₙOC(O)O(C₁-C₁₅) alkyl, —(CH₂)ₙSC(O)(C₁-C₁₅) alkyl, —(CH₂)ₙC(O)O(C₁-C₁₅) alkyl, and —(CH₂)ₙC(O)O(C₁-C₁₅)alkylaryl group; wherein n is an integer from 1-8; P(O)(OH)OP(O)(OH)₂. halogen, nitro, cyano, C₁-C₆ alkoxy, C₁-C₆ haloalkoxy, —N(R₁₁ₐ)₂, C₁-C₆ acylamino, —COR_{11ab}, —OCOR_{11b}; NHSO₂(C₁-C₆ alkyl), —SO₂N(R₁₁ₐ)₂SO₂; wherein each R₁₁ₐ is independently selected from H and C₁-C₆ alkyl, and R_{11b} is independently selected from OH, C₁-C₆ alkoxy, NH₂, NH(C₁-C₆ alkyl), and N(C₁-C₆ alkyl)₂;
R₁₂ is selected from H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the aryl or heteroaryl groups are optionally substituted with one or two groups selected from halogen, trifluoromethyl, C₁-C₆ alkyl, C₁-C₆ alkoxy, and cyano; and
R_{A} and R_{A′} are independently selected from H, C₁-C10 alkyl, C₂-C10 alkenyl, C₂-C10 alkynyl, C₃-C₁₀ cycloalkyl, C₁-C10 thioalkyl, C₁-C10 hydroxylalkyl, C₁-C10 alkylaryl, and C₅-C12 aryl, C₃-C10 heterocycloalkyl, heteroaryl, —(CH₂)₃NHC(═NH)NH₂, (1H-indol-3-yl) methyl, (1H-imidazol-4-yl) methyl, and a side chain selected from a proteinogenic or non-proteinogenic amino acid; wherein the aryl groups are optionally substituted with a group selected from hydroxyl, C₁-C10 alkyl, C₁-C6 alkoxy, halogen, nitro, and cyano; or
R₉ and R₁₀, together with the phosphorus atoms to which they are attached, form a 6-membered cycle, wherein —R₉-R₁₀— is —CH₂—CH₂—CHR—; wherein R is selected from H, a (C₅-C₆) aryl, and a (C₅-C₆) heteroaryl group; wherein the aryl or heteroaryl groups are optionally substituted with halogen, trifluoromethyl, C₁-C₆ alkyl, C₁-C6 alkoxy, and cyano; or
   R₉ and R₁₀ form, together with the phosphorus atoms to which they are attached, a 6-membered cycle, wherein —R₉-R₁₀— is —O—CH₂—CH₂—CHR—O—; wherein R is selected from H, a (C₅-C₆) aryl and a (C₅-C₆) heteroaryl group, wherein the aryl or heteroaryl groups are optionally substituted by a halogen, trifluoromethyl, a (C₁-C₆) alkyl, a (C₁-C₆) alkoxy, and cyano;
R₈ is selected from H, OR, NHR₁₃, NR₁₃R₁₄, NH—NHR₁₃, SH, CN, N₃, and halogen; wherein R₁₃ and R₁₄ selected, independently of one another, from H, (C₁-C₈) alkyl, (C₁-C₈) alkylaryl, and —CR_{B}R_{C}—C(O)—OR_{D}; wherein R_{B} and R_{C} are independently a hydrogen atom, (C₁-C₆) alkyl, (C₁-C₆) alkoxy, benzyl, indolyl or imidazolyl; wherein the (C₁-C₆) alkyl and the (C₁-C₆) alkoxy may, optionally and independently of one another, be substituted by one or more halogen, amino, amido, guanidyl, hydroxyl, thiol, or carboxyl groups; and the benzyl group is optionally substituted by one or more halogen or hydroxyl groups; or R_{B} and R_{C}. together with the carbon atom to which they are attached, form a C₃-C₆ cycloalkyl group optionally substituted with one or more halogen, amino, amido, guanidyl, hydroxyl, thiol, and carboxyl; and R_{D} is hydrogen, (C₁-C₆) alkyl, (C₂-C₆) alkenyl, (C₂-C₆) alkynyl, or (C₃-C₆) cycloalkyl;
Y is selected from CH, CH₂, C(CH₃)₂, and CCH₃;
[symbol] is a single or double bond depending on Y; and
[symbol] is the alpha or beta anomer depending on the position of R₁
   or
   a compound of formula (Ia):

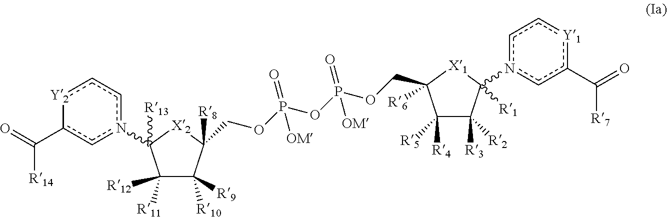

or a pharmaceutically acceptable stereoisomer, salt, hydrate, solvate, or crystal thereof, or a combination thereof, wherein
X′₁ and X′₂ are independently selected from O, CH₂, S, Se, CHF, CF₂, and C═CH₂;
R′₁ and R′₁₃ are independently selected from H, azido, cyano, C₁-C8 alkyl, C1-C8 thioalkyl, C1-C8 heteroalkyl, and OR; wherein R is selected from H and C1-C8 alkyl;
R′₂, R′₃, R′₄, R′₅, R′₉, R′₁₀, R′₁₁, R′₁₂ are independently selected from H, halogen, azido, cyano, hydroxyl, C₁-C₁₂ alkyl, C₁-C₁₂ thioalkyl, C₁-C₁₂ heteroalkyl, C₁-C₁₂ haloalkyl, and OR; wherein R may be selected from H, C₁-C₁₂ alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)(C₁-C₁₂) aryl, C(O)NH(C₁-C₁₂) alkylaryl, C(O)O(C₁-C₁₂) alkylaryl, and a C(O)CHR_{AA}NH₂ group; wherein R_{AA} is a side chain selected from the proteinogenic amino acids;
R′₆ and R′₈ are independently selected from H, azido, cyano, C₁-C₈ alkyl, and OR, wherein R is selected from H and C₁-C₈ alkyl;
R′₇ and R′₁₄ are independently selected from H, OR, NHR, NRR′, NH—NHR, SH, CN, N₃, and halogen; wherein R and R′ are independently selected from H and (C₁-C₈) alkylaryl;
Y′1 and Y′2 are independently selected from CH, CH₂, C(CH₃)₂ and CCH₃;
M′ is selected from H and a suitable counterion;
[symbol] is a single or double bond, depending on Y′1 and Y2; and [symbol] is an alpha or beta anomer depending on the position of R′₁ and R′₁₃;
   and combinations thereof.

In a first preferred embodiment, the pharmaceutically acceptable derivative is the compound of formula (I).

In one variant of the first embodiment, X is oxygen.

In one variant of the first embodiment, R₁ and R₆ each, independently of one another, are hydrogen.

In one variant of the first embodiment, R₂, R₃, R₄, and R₅ each, independently of one another, are hydrogen or OH.

In one variant of the first embodiment, Y is CH.

In one variant of the first embodiment, Y is CH₂.

In one variant of the first embodiment, R₇ is hydrogen.

In one variant of the first embodiment, R₇ is P(O)(OH)₂.

In one variant of the first embodiment,

X is oxygen; and/or

R₁ and R₆ are each independently hydrogen; and/or

R₂, R₃, R₄, and R₅ each independently are hydrogen, or R₂, R₃, R₄, and R₅ is independently OH; and/or

Y is CH or CH₂; and/or

R₇ is P(O)R₉R₁₀, wherein R₉ and R₁₀ are independently selected from OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, C₁-C₈ arylalkyl, C₁-C8 alkylaryl, C₁-C₈ heteroalkyl, C₁-C₈ heterocycloalkyl, heteroaryl, and NHCR_{A}R_{A′}C(O)R₁₂.

In a particularly preferred variant of the first embodiment, the compound of the invention is selected from compounds of formula I-A-I-J:

TABLE 1

| Compounds | |
|---|---|
| (anomers) | Structure |
| I-A (beta) | 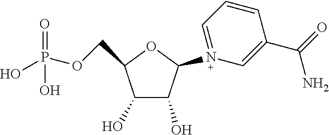 |
| I-B (alpha) | 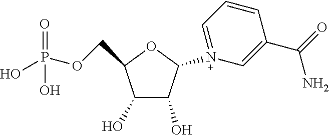 |
| I-C (beta) | 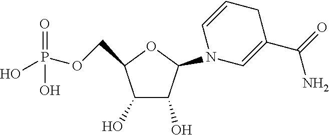 [SMILES: NC(=O)C1=CN([C@@H]2O[C@H](COP(=O)(O)O)[C@@H](O)[C@H]2O)C=CC1] |
| I-D (alpha) | 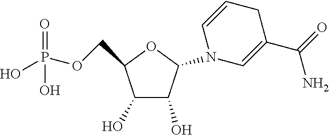 [SMILES: NC(=O)C1=CN([C@H]2O[C@H](COP(=O)(O)O)[C@@H](O)[C@H]2O)C=CC1] |
| I-E (beta) | 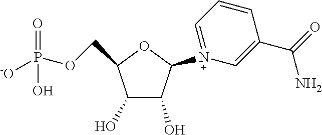 |
| I-F (alpha) | 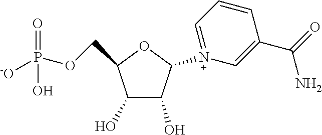 |
| I-G (beta) | 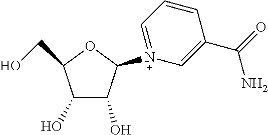 |
| I-H (alpha) | 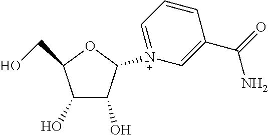 |
| I-I (beta) | 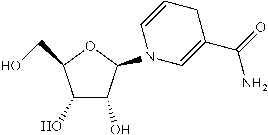 [SMILES: NC(=O)C1=CN([C@@H]2O[C@H](CO)[C@@H](O)[C@H]2O)C=CC1] |
| I-J (alpha) | 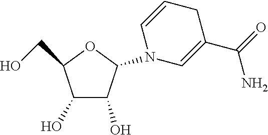 [SMILES: NC(=O)C1=CN([C@H]2O[C@H](CO)[C@@H](O)[C@H]2O)C=CC1] |

In a second preferred embodiment, the pharmaceutically acceptable derivative is the compound of formula (Ia).

In one variant of the second embodiment, X′1 and X′2 are each independently oxygen.

In one variant of the second embodiment, R′7 and R′14 are each independently NH2.

In one variant of the second embodiment, R′1 and/or R′13 are each independently hydrogen.

In one variant of the second embodiment, R′6 and/or R′8 are each independently hydrogen.

In one variant of the second embodiment, R′2, R′3, R′4, R′⁵, R′9, R′10, R′11, and R′12 are each independently hydrogen.

In one variant of the second embodiment, R′2, R′3, R′4, R′5. R′9, R′10, R′11, and R′12 are each independently OH.

In one variant of the second embodiment, Y′1 and Y′2 are each independently CH.

In one variant of the second embodiment, Y′1 and Y′2 are each independently CH2.

In one variant of the first embodiment, the compound of the invention is selected from compounds of formula Ia-A-Ia-I:

TABLE 2

| Compounds | |
|---|---|
| (anomers) | Structure |
| Ia-A (beta, beta) | 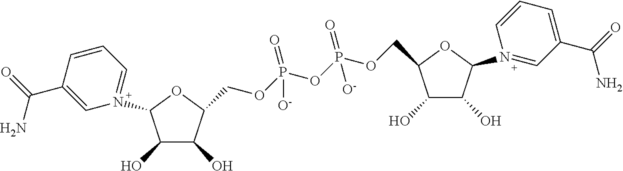 |
| Ia-B (beta, alpha) | |
| Ia-C (alpha, alpha) | |
| Ia-D (beta, beta) | |
| Ia-E (beta, alpha) | |
| Ia-F (alpha, alpha) | |
| Ia-G (beta, beta) | 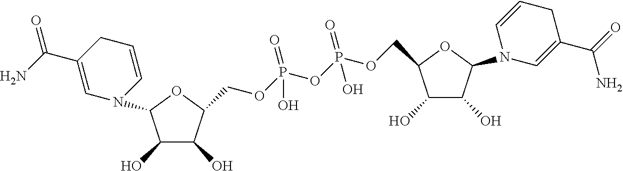 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | |
| Ia-I (alpha, alpha) | 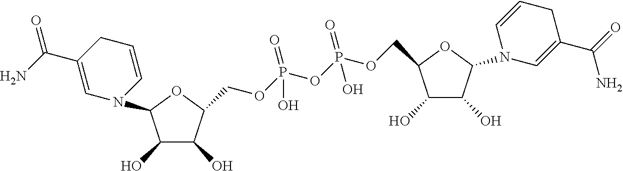 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

In one variant of the first preferred embodiment, the compound of formula (I) is the alpha-NMN of formula IF:

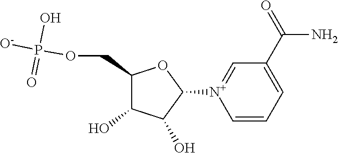 [SMILES: NC(=O)c1ccc[n+]([C@H]2O[C@H](COP(=O)([O-])O)[C@@H](O)[C@H]2O)c1]

In one variant of the first preferred embodiment, the compound of formula (I) is the NMN-H of formula I-D or IC.

In one variant of the first preferred embodiment, the compound of formula (I) is the nicotinamide riboside (‘NR’) of formula I-G or 1-H, or the dihydronicotinamide riboside (‘—NR—H’) of formula I-J or I-I.

Preferably, the compound of formula (I) is selected from compound I-A, compound I-B, compound I-C, compound I-D, compound I-E, compound I-F, compound I-G, compound I-H, compound I-I, compound I-J, preferably compound I-C, compound I-D, or compound I-F, and combinations thereof. More preferably, the compound of formula (I) is selected from compound IB, compound IC, compound ID, compound IF, and combinations thereof.

Preferably, the compound of formula (Ia) is selected from the compounds of formula Ia-A-Ia-I, more preferably from the compound of formula Ia-B, the compound of formula Ia-C, the compound of formula Ia-E, the compound of formula Ia-F, the compound of formula Ia-H, the compound of formula Ia-I, and the compound of formula Ia-G, as well as combinations thereof.

Advantageously, the compound of formula (I) or the compound of formula (Ia) may be used to prevent or treat the peripheral form of AS.

Advantageously, the compound of formula (I) or the compound of formula (Ia) may be used to prevent or treat the axial form of AS.

Advantageously, the compound of formula (I) or the compound of formula (Ia) may be used in an amount between 0.01 mg/kg/d and 1000 mg/kg/d, preferably 1 mg/kg/d and 100 mg/kg/d, more preferably between 5 mg/kg/d and 50 mg/kg/d, even more preferably between 10 mg/kg/d and 20 mg/kg/d.

Advantageously, the compound of formula (I) or the compound of formula (Ia) may be administered orally, intraocularly, sublingually, intravenously, intraarterially, intramuscularly, intraarticularly, subcutaneously, transcutaneously, vaginally, peridurally, intravesically, rectally, or by inhalation.

In a preferred embodiment, the compound of formula (I) or the compound of formula (Ia) may be administered orally.

In a more preferred embodiment, the compound of formula (I) or the compound of formula (Ia) may be administered in the form of a sublingual tablet or a gastroresistant capsule.

In a preferred alternative embodiment, the compound of formula (I) or the compound of formula (Ia) may be administered intraarticularly.

Advantageously, the compound of formula (I) or the compound of formula (Ia) may be used in the treatment and/or prevention of AS in mammals, preferably humans.

Advantageously, the compound of formula (I) or the compound of formula (Ia) may be used in combination with at least one additional therapeutic agent.

Advantageously, the at least one additional therapeutic agent may be from an analgesic, an NSAID, cortisone, a cortisone derivative, an immunosuppressant, an immunomodulator, an anti-TNF agent, an anti-interleukin agent, and combinations thereof.

Advantageously, the analgesic may be selected from paracetamol, aspirin, codeine, dihydrocodeine, tramadol, morphine, buprenorphin, fentanyl, hydromorphone, nalbuphine, oxycodone, pethidine, and combinations thereof.

Advantageously, the NSAID may be selected from ibuprofen, ketoprofen, naproxen, alminoprofen, aceciofenac, mefenamic acid, niflumic acid, tiaprofenic acid, celecoxib, dexketoprofen, diclofenac, etodolac, etoricoxib, fenoprofen, flurbiprofen, indomethacin, meloxicam, nabumetone, piroxicam, sulindac, tenoxicam, and combinations thereof.

Advantageously, the cortisone derivative may be selected from betamethasone, ciprofloxacin, cortivazol, dexamethasone, fludrocortisone, methylprednisolone, prednisolone, triamcinolone, and combinations thereof.

Advantageously, the immunosuppressant may be selected from azathioprine, cyclophosphamide, chlorambucil, cyclosporine, methotrexate, and combinations thereof.

In a preferred embodiment, the immunosuppressant may be methotrexate or cyclosporine, more preferably methotrexate.

Advantageously, the immunomodulator may be selected from leflunomide, sulphasalazine, and combinations thereof, preferably sulphasalazine.

Advantageously, the anti-TNF agent may be selected from infliximab, etanercept, adalimumab, certolizumab, golimumab, and combinations thereof.

Advantageously, the anti-interleukin agent may be an anti-interleukin 17.

Advantageously, the interleukin 17 inhibitor may be selected from ixekizumab and secukinumab.

Advantageously, the anti-interleukin agent may be an anti-interleukin 12, preferably ustekinumab.

This invention also concerns a composition comprising a compound of formula (I) as defined herein and/or a compound of formula (Ia) as defined herein and at least one pharmaceutically acceptable excipient for use in the prevention and/or treatment of ankylosing spondylitis.

Preferably, the compound of formula (I) is selected from compound I-A, compound I-B, compound I-C, compound I-D, compound I-E, compound I-F, compound I-G, compound I-H, compound I-I, compound I-J, preferably compound I-C, compound I-D, or compound I-F, and combinations thereof. More preferably, the compound of formula (I) is selected from compound IB, compound IC, compound ID, compound IF, and combinations thereof.

Preferably, the compound of formula (Ia) is selected from the compounds of formula Ia-A-Ia-I, more preferably from the compound of formula Ia-B, the compound of formula Ia-C, the compound of formula Ia-E, the compound of formula Ia-F, the compound of formula Ia-H, the compound of formula Ia-I, and the compound of formula Ia-G, as well as combinations thereof.

Preferably, the composition according to the invention further comprises at least one additional therapeutic agent as defined supra.

Advantageously, the composition according to the invention may be provided in the form of a tablet, capsule, sachet, granulate, soft capsule, lyophilisate, suspension, gel, syrup, solution, water-in-oil emulsion, oil-in-water emulsion, oil, cream, milk, spray, ointment, ampule, suppository, eye drops, vaginal ovule, vaginal capsule, liquid for inhalation, dry powder inhaler, pressurised metered-dose inhaler.

Advantageously, the composition according to the invention may be a pharmaceutical composition.

Advantageously, the composition according to the invention may be a dietary supplement.

Advantageously, the composition according to the invention may be administered orally, intraocularly, sublingually, intravenously, intramuscularly, intraarticularly, subcutaneously, transcutaneously, vaginally, peridurally, intravesically, rectally, or by inhalation.

In a preferred embodiment, the composition according to the invention may be administered orally.

In a more preferred embodiment, the composition according to the invention may be administered in the form of a sublingual tablet or a gastroresistant capsule.

In a preferred embodiment, the composition according to the invention may be administered by injection, preferably intraarticular injection.

In a preferred embodiment, the composition according to the invention may further comprise at least one additional therapeutic agent as defined supra for use in the prevention and/or treatment of AS as described supra.

Another object of the invention is a kit comprising a compound of formula (I) as defined supra, and/or a compound of formula (Ia) as defined supra, and/or a composition according to the invention as defined supra and at least one additional therapeutic agent for use in the prevention and/or treatment of AS as described supra.

### Definitions

In this invention, the following terms have the following meanings.

Unless otherwise indicated, the nomenclature of the substituents that are not explicitly defined in this invention is obtained by naming the terminal part of the functionality followed by the adjacent functionality in the direction of the point of attachment. ‘Alkyl’ on its own or as part of another substituent refers to a hydrocarbyl radical having the formula CnH2n+1, wherein n is a number greater than or equal to 1. In general, the alkyl groups of this invention comprise 1-12 carbon atoms, preferably 1-8 carbon atoms, more preferably 1-6 carbon atoms, even more preferably 1-2 carbon atoms. Alkyl groups may be linear or branched, and may be substituted as indicated in this invention. Alkyls suitable for the implementation of the invention may be selected from methyl, ethyl, n-propyl, i-propyl, n-butyl, i-butyl, s-butyl, and t-butyl, pentyl and isomers thereof such as n-pentyl and iso-pentyl, hexyl and isomers thereof such as n-hexyl and iso-hexyl, heptyl and isomers thereof (e.g. n-heptyl, iso-heptyl), octyl and isomers thereof (e.g. n-octyl, iso-octyl), nonyl and isomers thereof (e.g. n-nonyl, iso-nonyl), decyl and isomers thereof (e.g. n-decyl, iso-decyl), undecyl and isomers thereof, dodecyl and isomers thereof. Preferably, alkyl groups may be selected from methyl, ethyl, n-propyl, i-propyl, n-butyl, i-butyl, s-butyl, t-butyl, n-pentyl, n-hexyl, n-heptyl, n-octyl, n-nonyl, and n-decyl. Saturated and branched alkyl groups may be selected, without limitation, from isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-17 ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, and 3,3-diethylhexyl. Preferred are the following alkyl groups: methyl, ethyl, n-propyl, i-propyl, n-butyl, i-butyl, s-butyl, and t-butyl. Cx-Cy alkyls refer to alkyl groups comprising x-y carbon atoms.

When the suffix ‘ene’ (‘alkylene’) is used in conjunction with an alkyl group, this means that the alkyl group as defined herein has two single bonds as points of attachment to other groups. The term ‘alkylene’ includes methylene, ethylene, methylmethylene, propylene, ethylethylene, and 1,2-dimethylethylene.

As used herein, the term ‘alkenyl’ refers to an unsaturated hydrocarbyl group that may be linear or ramified and comprises one or more carbon-carbon double bonds. Suitable alkenyl groups comprise between 2 and 12 carbon atoms, preferably between 2 and 8 carbon atoms, and even more preferably between 2 and 6 carbon atoms. Examples of alkenyl groups include ethenyl, 2-propenyl, 2-butenyl, 3-butenyl, 2-pentenyl and isomers thereof, 2-hexenyl and isomers thereof, 2,4-pentadienyl, and similar groups.

As used herein, the term ‘alkynyl’ refers to a class of monovalent unsaturated hydrocarbyl groups, in which the unsaturation arises from the presence of one or more carbon-carbon triple bonds. Generally, and preferably, alkynyl groups have the same number of carbon atoms as described supra for alkenyl groups. Examples of alkynyl groups include, without limitation, ethynyl, 2-propynyl, 2-butynyl, 3-butynyl, 2-pentynyl and isomers thereof, 2-hexynyl and isomers thereof, etc.

‘Alkoxy’ refers to an alkyl group as defined supra that is attached to another part by an oxygen atom. Examples of alkoxy groups include, inter alia, methoxy, isopropoxy, ethoxy, and tert-butoxy groups. Alkoxy groups may be optionally substituted by one or more substituents. Alkoxy groups included in the compounds of this invention may optionally be substituted with a solubilising group.

As used herein, ‘aryl’ refers to a polyunsaturated aromatic hydrocarbyl group having a single cycle (e.g. phenyl) or several aromatic cycles fused together (e.g. naphthyl) or covalently bonded, generally containing 5-18 atoms, preferably 5-12, more preferably 6-10, at least one of which cycles is aromatic. The aromatic cycle may optionally comprise one or two additional cycles (cycloalkyl, heterocyclyl, or heteroaryl) fused with it. ‘Aryl’ is also intended to include partially hydrogenated derivatives of the carbocyclic systems set forth herein. Examples of aryl include phenyl, biphenylyl, biphenylenyl, 5- or 6-tetralinyl, naphtalene-1- or -2-yl, 4-, 5-, 6 or 7-indenyl, 1-2-, 3-, 4- or 5-acenaphthylenyl, 3-, 4- or 5-acenaphtenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl.

If at least one carbon atom in an aryl group is replaced by a heteroatom, the resultant cycle is referred to herein as a ‘heteroaryl’ cycle.

‘Alkylaryl’ refers to an aryl group substituted with an alkyl group.

‘Amino acid’ refers to an alpha-amino carboxylic acid, i.e. a molecule comprising a carboxylic acid functional group and an amino functional group in the alpha position of the carboxylic acid group, e.g. a proteinogenic amino acid or a non-proteinogenic amino acid.

‘Proteinogenic amino acid’ refers to an amino acid incorporated into proteins in the translation of messenger RNA (mRNA) by ribosomes in living organisms, i.e. Alanine (ALA), Arginine (ARG), Asparagine (ASN), Aspartate (ASP), Cysteine (CYS), Glutamate (glutamic acid) (GLU), Glutamine (GLN), Glycine (GLY), Histidine (HIS), Isoleucine (ILE), leucine (LEU), Lysine (LYS), Methionine (MET), Phenylalanine (PHE), Proline (PRO), Pyrrolysine (PYL), Selenocysteine (SEL), Serine (SER), Threonine (THR), Tryptophane (TRP), Tyrosine (TYR), or Valine (VAL).

As used herein, ‘non-proteinogenic amino acid’ refers to an amino acid that is not naturally encoded or found in the genetic code of a living organism. Examples of non-proteinogenic amino acids include, without limitation, omithine, citrulline, argininosuccinate, homoserine, homocysteine, cysteine-sulphinic acid, 2-aminomuconic acid, δ-aminolevulinic acid, β-alanine, cystathionine, γ-aminobutyrate, DOPA, 5-hydroxytryptophan, D-serine, ibotenic acid, α-aminobutyrate, 2-aminoisobutyrate, D-leucine, D-valine, D-alanine, or D-glutamate.

As used herein, the term ‘cycloalkyl’ is a cyclic alkyl group, i.e. a saturated or unsaturated monovalent hydrocarbyl group having 1 or 2 cyclic structures. The term ‘cycloalkyl’ includes monocyclic or bicyclic hydrocarbyl groups. Cycloalkyl groups may comprise 3 or more carbon atoms in the cycle and, generally, according to this invention, may comprise 3-10, more preferably 3-8 carbon atoms, and even more preferably 3-6 carbon atoms. Examples of cycloalkyl groups include, without limitation, cyclopropyl, cyclobutyl, cyclopentyl, cyclohexyl, with cyclopropyl being particularly preferred.

‘Pharmaceutically acceptable excipient’ refers to an inert vehicle or support used as a solvent or diluent in which the active ingredient is formulated and/or administered, and that does not produce any undesirable, allergic, or other reaction when administered to an animal, preferably a human. This includes all solvents, dispersion media, coatings, antibacterials and antifungals, isotonic agents, absorption retardants, and other similar ingredients. For administration to humans, the preparations must meet sterility, general safety, and purity standards imposed by regulatory bodies such as the FDA or the EMA. Within the meaning of the invention ‘pharmaceutically acceptable excipient’ includes all pharmaceutically acceptable excipients, as well as all pharmaceutically acceptable supports, diluents, and/or adjuvants.

‘Halogen’ or ‘halo’ means fluoro, chloro, bromo, or iodo. Preferred halo groups are fluoro and chloro.

‘Haloalkyl’, alone or in combination, refers to an alkyl radical within the meaning set forth supra, wherein one or more hydrogen atoms are replaced by a halogen as defined supra. Examples of such haloalkyl radicals include chloromethyl, 1-bromoethyl, fluoromethyl, difluoromethyl, trifluoromethyl, 1,1,1-trifluoroethyl, and similar radicals. Cx-Cy-haloalkyl and Cx-Cy-alkyl refer to alkyl groups comprising x-y carbon atoms. Preferred haloalkyl groups are difluoromethyl and trifluoromethyl.

‘Heteroalkyl’ refers to an alkyl group as defined supra, wherein one or more carbon atoms are replaced by a heteroatom selected from oxygen, nitrogen, and sulphur atoms. In heteroalkyl groups, the heteroatoms are bonded along the alkyl chain only to carbon atoms, i.e. each heteroatom is separated from every other heteroatom by at least one carbon atom. However, nitrogen and sulphur atoms may be optionally oxidised, and nitrogen heteroatoms may optionally be quarternised. A heteroalkyl is bonded to another group or molecule only by a carbon atom, i.e. the bonding atom is not selected from the heteroatoms included within the heteroalkyl group.

As used herein, the term ‘heteroaryl’, whether alone or as part of another group, refers, without limitation, to aromatic cycles having 5-12 carbon atoms or cyclic systems containing 1 or 2 cycles that are fused or covalently bonded, generally containing 5 or 6 atoms, at least one of which is aromatic, wherein one or more carbon atoms in one or more of these cycles are replaced by oxygen, nitrogen, and/or sulphur atoms, wherein the nitrogen and sulphur heteroatoms may optionally be oxidised and the nitrogen heteroatoms may optionally be quarternised. These cycles may be fused with an aryl, cycloalkyl, heteroaryl, or heterocyclyl cycle. Examples of such heteroaryl groups include, without limitation, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo [2, 1-b] [1,3] thiazolyl, thieno [3,2-b] furanyl, thieno [3,2-b] thiophenyl, thieno [2,3-d] [I,3] thiazolyl, thieno [2,3-d] imidazolyl, tetrazolo [I,5-a] pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-I(6H)-yl, 2-oxopyridin-I(2H)-yl, 6-oxo-pyridazin-I(6H)-yl, 2-oxopyridin-I(2H)-yl, 1,3-benzodioxolyl, quinolinyl, isoquinolinyl, cinnolinyl, quinazolinyl, quinoxalinyl.

If at least one carbon atom in a cycloalkyl group is replaced by a heteroatom, the resultant cycle is referred to herein as ‘heterocycloalkyl’ or ‘heterocyclyl’.

As used herein, the terms ‘heterocyclyl’, ‘heterocycloalkyl’, or ‘heterocyclo’, alone or as part of another group, refer to non-aromatic cyclic groups that are totally saturated or partially unsaturated, (e.g. monocyclic with 3-7 members, bicyclic with 7-11 members, or containing a total of 3-10 cycle atoms) having at least one heteroatom in at least one cycle containing a carbon atom. Each cycle of the heterocyclic group containing a heteroatom may have 1, 2, 3, or 4 heteroatoms selected from nitrogen, oxygen, and/or sulphur atoms, wherein the nitrogen and sulphur heteroatoms may optionally be oxidized and the nitrogen heteroatoms may optionally be quaternised. Any carbon atom of the heterocyclic group may be substituted by an oxo (e.g. piperidone, pyrrolidinone). The heterocyclic group may be attached to any heteroatom or carbon atom of the cycle or cyclic system if the valence permits. The cycles of the multicyclic heterocycles may be fused, bridged, and/or bonded by one or more spiro atoms. Non-limiting examples of heterocyclic groups include oxetanyl, piperidinyl, azetidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2, 5-dioximidazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinoline-4-yl [sic], thiomorpholine-4-yl, thiomorpholine-4-ylsulph [sic] oxide, thiomorpholine-4-ylsulphone, 1,3-dioxolanyl, 1,4-oxathianyl, 1H-pyrrolizinyl, tetrahydro-I,I-dioxothiophenyl, N-formylpiperazinyl, and morpholine-4-yl.

As used herein, ‘precursor’ also refers to pharmaceutically acceptable derivatives of the compounds of formula (I) or (Ia), such as esters, the in vivo biotransformation project of which is the active drug. Precursors are characterised by increased bioavailability, and they are easily metabolised into active compounds in vivo. Precursors suited to the purposes of the invention include, without limitation, carboxylic esters, in particular alkylic esters, arylic esters, acyloxyalkylic esters and carboxylic esters of dioxolene; ascorbic acid esters.

‘Pharmaceutically acceptable’ means approved or eligible for approval by a regulatory body or registered in a recognised pharmacopoeia for use in animals, and more preferably in humans. This may be a substance that is not undesirable in biological or other terms, i.e. the substance may be administered to an individual without causing undesirable biological effects or deleterious interactions with one of the components of the composition in which it is contained. Preferably, a ‘pharmaceutically acceptable’ salt or excipient refers to any salt or excipient authorized by the European Pharmacopoeia (‘Ph. Eur.’) and the United States Pharmacopeia (‘USP’).

‘Active ingredient’ or ‘therapeutic (agent)’ refers to a molecule or substance, the administration of which to a subject slows or stops the progression, worsening, or deterioration of one or more symptoms of a disease or condition, relieves the symptoms of a disease or condition, or cures a disease or condition. In one of these embodiments, the therapeutic ingredient is a small molecule, natural or synthetic. In another, the therapeutic ingredient is a biological molecule, e.g. an oligonucleotide, siRNA, miRNA, DNA fragment, aptamer, antibody, etc. ‘Pharmaceutically acceptable salts’ include acid and base addition salts of these salts. Suitable acid addition salts are formed from acids that form non-toxic salts. These include, for example, acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, chlorhydrate/chlorure, bromhydrate/bromure, hydroiodure/iodure, isethionate, lactate, malate, maleate, malonate, mesylate, methylsulphate, naphthylate, 2-napsylate, nicotinate, nitrate, orotate, oxalate, palmitate, pamoate, phosphate/hydrogenophosphate/dihydrogenophosphate, pyroglutamate, saccharate, stearate, succinate, tannate, tartrate, tosylate, trifluoroacetate, and xinofoate salts. Suitable basic salts are formed from bases that form non-toxic salts. Examples include aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine, and zinc salts. Hemisalts of acids and bases may also be formed, e.g. hemisulphates and chemical calcium salts. Preferred pharmaceutically acceptable salts are chlorhydrate/chloride, bromide/hydrobromide, bisulphate/sulphate, nitrate, citrate, and acetate.

Pharmaceutically acceptable salts may be prepared by one or more of the following methods:
i. by reacting the compound with the desired acid;
ii. by reacting the compound with the desired base;
iii. by eliminating an acid- or base-labile protecting group of a suitable precursor of the compound or by opening the cycle of a suitable cyclic precursor, e.g. a lactone or lactam, using the desired acid; or
iv. by transforming a salt of the compound into another by reacting it with a suitable acid or by means of a suitable ion exchange column.

All these reactions are generally carried out in solution. The salt may precipitate from the solution and be collected by filtration, or it may be recovered by evaporating the solvent. The degree of ionisation of the salt may vary from completely ionised to nearly non-ionised.

‘Solvate’ is used herein to describe a molecular complex comprising the compound of the invention and one or more pharmaceutically acceptable solvent molecules, e.g. ethanol.

The term ‘substituent’ or ‘substituted’ means that a hydrogen radical on a compound or group is replaced by any desired group that is substantially stable in the reaction conditions in a non-protected form or when protected by a protecting group. Examples of preferred substituents include, without limitation, halogen (chloro, iodo, bromo, or fluoro); alkyl; alkenyl; alkynyl, as described supra; hydroxy; alkoxy; nitro; thiol; thioether; imine; cyano; amido; phosphonato; phosphine; carboxyl; thiocarbonyl; sulphonyl; sulphonamide; ketone; aldehyde; ester; oxygen (—O); haloalkyl (e.g. trifluoromethyl); cycloalkyl, which may be monocyclic or polycyclic, condensed or non-condensed (e.g., cyclopropyl, cyclobutyl, cyclopentyl, or cyclohexyl), or heterocycloalkyl, which may be monocyclic or polycyclic, condensed or non-condensed (e.g., pyrrolidinyl, piperidinyl, piperazinyl, morpholinyl, or thiazinyl), monocyclic or polycyclic, fused or unfused, aryl or heteroaryl (e.g., aryl, heteroaryl, pyrrolidinyl, piperidinyl, piperazinyl, morpholinyl, or thiazinyl), monocyclic or polycyclic, fused or unfused (e.g., aryl, heteroaryl, pyrrolidinyl, piperidinyl, piperazinyl, morpholinyl, or thiazinyl), phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH2; OCH₂CONH₂; NH₂; SO₂NH₂; OCHF₂; CF₃; OCF₃; and these groups may also be optionally substituted by a structure or fused annular bridge, e.g. —OCH₂O—. These substituents may also optionally be substituted with a substituent chosen from these groups. In certain representations, the term ‘substituent’ or the adjective ‘substituted’ refers to a substituent selected from the group consisting of alkyl, alkenyl, alkynyl, cycloalkyl, cycloalkenyl, heterocycloalkyl, aryl, heteroaryl, arylalkyl, heteroarylalkyl, haloalkyl, —C(O)NR₁₁R₁₂, —NR₁₃C(O)R₁₄, halo, —OR₁₃, cyano, nitro, un haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —N—R₁₃, wherein r is 1 or 2; R₁₁ and R₁₂, in each occurrence, are independently H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, or optionally substituted heteroarylalkyl; or R₁₁ and R₁₂, taken together with the nitrogen to which they are attached, are optionally substituted heterocycloalkyl or optionally substituted heteroaryl; and R13 and R14, in each occurrence, are independently H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, or optionally substituted heteroarylalkyl. In certain variants, the term ‘substituent’ or the adjective ‘substituted’ refers to a solubilising group.

The term ‘administration’ or any variant thereof (e.g. ‘to administer’) means providing the active ingredient, alone or within a pharmaceutically acceptable composition, to a patient in need of treatment or prevention of the condition, symptom, or disease.

‘To treat’, ‘to care’, and ‘treatment’, as used herein, are intended to include relieving, attenuating, or eliminating a condition or disease and/or the associated symptoms.

‘To prevent’ and ‘prevention’, as used herein, refer to a method allowing for the appearance of a condition or disease and/or its associated symptoms to be delayed or prevented, for a patient to be prevented from contracting a condition or disease, or for a reduction in a patient's risk of contracting a condition or disease.

Asymmetrical carbon bonds may be represented here using an unbroken triangle ( [symbol] ), a dotted triangle ( [symbol] ), or a zigzag line ( [symbol] ).

### DETAILED DESCRIPTION OF THE INVENTION

This invention concerns the compound of formula (I) and/or the compound of formula (Ia) for use in the prevention and/or treatment of AS, as well as compositions comprising it.

Nicotinamide adenine dinucleotide (NAD) is a coenzyme present in all living cells. NAD exists in the cell either in its oxidised form, NAD+, or in the reduced form, NADH. The role of NAD is as a transporter of electrons involved in metabolic redox reactions. NAD is also involved numerous cellular processes, such as the ribosylation of ADP in post-translational protein modifications.

NAD may be synthesised de novo by the cell from amino acids such as tryptophan or aspartate. However, this synthesis is marginal, because the main synthesis pathway of NAD is that of salvage, by which the cell, and mainly the cell nucleus, recycles compounds to re-form NAD from precursors. NAD precursors include niacin, nicotinamide riboside, nicotinamide mononucleotide, and nicotinamide.

NMN is one of the compounds allowing for NAD synthesis via salvage, and has the formula I-E:

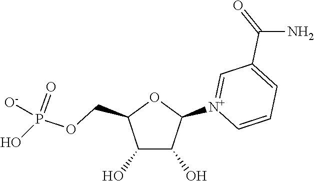 [SMILES: NC(=O)c1ccc[n+]([C@@H]2O[C@H](COP(=O)([O-])O)[C@@H](O)[C@H]2O)c1]

The inventors have in fact shown that the compound of formula (I) or (Ia), as well as the composition according to the invention, had an effect on the joint swelling caused by AS without having undesirable effects.

More specifically, the inventors found that compounds of formula (I) or (Ia), as well as compositions comprising them, made it possible to treat the inflammatory eruptions characteristic of AS by significantly reducing joint swelling, spinal column inflammation, and pelvic ankylosis. Moreover, the chronic administration of NMN allows for prevention, or at least increasing the intervals between, these eruptions. In fact, when administered chronically between each eruption, the compound of formula (I) or (Ia) and compositions comprising them are able to reduce inflammation and thus to prevent, or at least increase the intervals between, AS eruptions. The compounds and compositions according to the invention are particularly effective for treating the axial form and the peripheral form of AS.

Moreover, NMN, a molecule that is naturally present in the body, has numerous advantages. In particular, NMN does not raise any tolerance issues for patients. Indeed, the use of NMN and the composition according to the invention does not induce any allergic reaction. Moreover, the use of NMN and the composition according to the invention does not cause the side-effects frequently encountered with conventional treatments. The compounds of formula (I) or (Ia), which are structurally similar to NMN, have the same advantages.

In particular, the compounds of formula (I) or (Ia) et the compositions according to the invention do not cause any physical or psychological dependency, unlike analgesics comprising morphine or opium derivatives. In addition, the compounds of formula (I) or (Ia) and the compositions according to the invention do not cause the bone fragility or vulnerability to infection observed with chronic administration of cortisone or derivatives thereof. Thus, the use of compounds of formula (I) or (Ia) and the compositions according to the invention to prevent and/or treat AS is safe for patients.

The compounds of formula (I) or (Ia) and the compositions according to the invention may be used both in children and adults. Indeed, they are well tolerated by children. In the context of the invention, a patient is considered a child if their age is less than 18, and an adult starting at age 18. Ass such, the invention is also of interest for treating AS in children.

In a more preferred embodiment, the compounds of formula (I) or (Ia) are in the form of a zwitterion. A ‘zwitterion’ is a chemical molecular species having opposite electrical charges that are generally located on non-adjacent atoms of the molecule.

Firstly, the use of compounds of formula (I) or (Ia) and the compositions according to the invention makes it possible to treat inflammation during AS eruptions, as well as the sensation of spinal column stiffening.

By reducing inflation, and, in particular, swelling, in joints, as well as preventing inflammatory eruptions, it is also possible to reduce the pain related to the inflammation and to reduce joint stiffening. Thus, it is possible to avoid administering, or at least to reduce the frequency of administration and the dosage of, drugs used to combat the symptoms of ankylosing spondylitis, i.e. analgesics, NSAIDs, cortisone, and/or cortisone and derivatives thereof. This also makes it possible to avoid the administration of treatments that are conventionally used to treat AS, such as methotrexate, or at least to reduce their frequency of administration or dosage.

By reducing the need for conventional therapies, or even replacing them, this invention thus makes it possible to avoid, or at least reduce, the use of conventional AS treatments, and thus to avoid, or at least reduce, the appearance of side-effects connected to these treatments.

In addition to the therapeutic aspect, therefore, the invention makes it possible to maintain patients' quality of life by making it easier for them to carry out everyday tasks, and possibly avoiding the need to end professional activities. Thus, the invention serves to maintain, or at least to avoid excessive deterioration of, patient quality of life.

Use

According to this invention, the compounds of formula (I) or (Ia) and the compositions according to the invention are used to prevent and/or treat ankylosing spondylitis. More precisely, they may be used acutely to treat an eruption of AS or chronically to reduce inflammation and increase the intervals between eruptions. In other words, the compounds of formula (I) or (Ia) and the compositions according to the invention may be used for preventive or curative purposes, in order to reduce the inflammation, and, in particular, the joint swelling, in the spinal column and the pelvic ankylosis that are characteristic of the peripheral and axial forms of AS.

The compounds of formula (I) or (Ia) and the compositions according to the invention may be administered in a therapeutically effective amount. In the context of the invention, a therapeutically effective amount means that the composition is administered to a patient in an amount sufficient to obtain the desired therapeutic effect.

In one embodiment, the compounds of formula (I) or (Ia) may be used in an amount between 0.01 mg/kg/d and 1000 mg/kg/d, preferably 1 mg/kg/d and 100 mg/kg/d, more preferably between 5 mg/kg/d and 50 mg/kg/d, even more preferably between 10 mg/kg/d and 20 mg/kg/d. Persons skilled in the art are able to adapt the dose of NMN to be administered based on the age and weight of the patient, as well as the intensity of the pain being treated.

A suitable dosage level may be approximately 0.01-250 mg/kg/d, approximately 0.05-100 mg/kg/d, or approximately 0.1 à 50 mg/kg/d. Within this range, the dose may be 0.05-0.5, 0.5-5, or 5-50 mg/kg/d. For oral administration, the compositions are preferably provided in the form of tablets containing 1.0-1000 mg of the active ingredient, in particular 1.0 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 mg of active ingredient for symptom-based adjustment of the dose of the patient to be treated. For example, the dosage may be between 100 mg/d and 5000 mg/d, preferably between 500 mg/d and 1000 mg/d. The compounds may be administered according to a regimen of 1-4 administrations per day, preferably once, twice, or thrice a day, preferably thrice a day. The duration of treatment depends, and is determined by, the treating physician. It may range from one day to one year or more, preferably from one week to three months, more preferably from two weeks to six weeks. However, it is understood that the specific dosage level and frequency, as well as the duration for a given patient may vary, and will depend on various factors, in particular the activity of the specific compound used, the metabolic stability and duration of action of the compound, the age, body weight, general condition, sex, diet, mode and time of administration, excretion rate, combination of drugs, and the individual being treated.

The compounds of formula (I) or (Ia) and the compositions according to the invention may be administered once a day or several times a day. In particular, the compounds of formula (I) or (Ia) and the compositions according to the invention may be administered between 1 and 12 times a day, preferably between 2 and 10 times a day, more preferably between 3 and 5 times a day.

The dose administered and the frequency of administration depend, inter alia, on the stage of development of the inflammation. They may also depend on various factors, such as the weight, age, and sex of the patient.

Compounds of Formula (I) and (Ia):

In particular, this invention concerns the compound of formula (I):

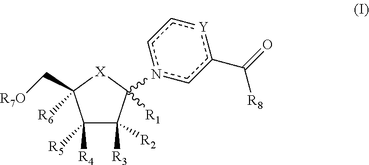

or a pharmaceutically acceptable stereoisomer, salt, hydrate, solvate, or crystal thereof, wherein
X is selected from O, CH₂, S, Se, CHF, CF₂, C═CH₂;
R₁ is selected from H, azido, cyano, C₁-C8 alkyl, C₁-C8 thioalkyl, C₁-C8 heteroalkyl, and OR; wherein R is selected from H and C₁-C8 alkyl;
R₂, R₃, R₄, and R₅ are selected independently of one another from H, halogen, azido, cyano, hydroxyl, C₁-C12 alkyl, C₁-C12 thioalkyl, C₁-C12 heteroalkyl, C₁-C12 haloalkyl, and OR; wherein R is selected from H, C₁-C₁₂ alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)NH(C₁-C₁₂) alkylaryl, C(O)O(C₁-C₁₂) alkylaryl, and C(O)CHR_{AA}NH₂; wherein R_{AA} is a side chain selected from the proteinogenic amino acids;
R₆ is selected from H, azido, cyano, C₁-C8 alkyl, C₁-C8 thioalkyl, C₁-C8 heteroalkyl, and OR; wherein R is selected from H and C₁-C8 alkyl;
R₇ is selected from H, P(O)R₉R₁₀, and P(S)R₉R₁₀; wherein
R₉ and R₁₀ are selected independently of one another from OH, OR₁₁, NHR₁₃, NR₁₃R₁₄, C₁-C₈ alkyl, C₂-C₈ alkenyl, C₂-C₈ alkynyl, C₃-C10 cycloalkyl, C₅-C12 aryl. (C₁-C₈) alkylaryl, (C₁-C₈) arylalkyl, (C₁-C₈) heteroalkyl, (C₁-C₈) heterocycloalkyl, heteroaryl, and NHCHR_{A}R_{A′}C(O)R₁₂; wherein:
R₁₁ is selected from a C₁-C10 alkyl, C₃-C10 cycloalkyl, C₅-C18 aryl, C₁-C10 alkylaryl, substituted C₅-C12 aryl, C₁-C10 heteroalkyl, C₃-C10 heterocycloalkyl, C₁-C₁₀ haloalkyl, heteroaryl, —(CH₂)ₙC(O)(C₁-C₁₅) alkyl, —(CH₂)ₙOC(O)(C₁-C₁₅) alkyl, —(CH₂)ₙOC(O)O(C₁-C₁₅) alkyl, —(CH₂)ₙSC(O)(C₁-C₁₅) alkyl, —(CH₂)ₙC(O)O(C₁-C₁₅) alkyl, and —(CH₂)ₙC(O)O(C₁-C₁₅)alkylaryl group; wherein n is an integer from 1-8; P(O)(OH)OP(O)(OH)₂. halogen, nitro, cyano, C₁-C₆ alkoxy, C₁-C₆ haloalkoxy, —N(R₁₁ₐ)₂, C₁-C₆ acylamino, —COR_{11b}, —OCOR_{11b}; NHSO₂(C₁-C₆ alkyl), —SO₂N(R₁₁ₐ)₂SO₂; wherein each R₁₁ₐ is independently selected from H and C₁-C₆ alkyl, and R_{11b} is independently selected from OH, C₁-C₆ alkoxy, NH₂, NH(C₁-C₆ alkyl), and N(C₁-C₆ alkyl)₂;
R₁₂ is selected from H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the aryl or heteroaryl groups are optionally substituted with one or two groups selected from halogen, trifluoromethyl, C₁-C₆ alkyl, C₁-C₆ alkoxy, and cyano; and
R_{A} and R_{A′} are independently selected from H, C₁-C10 alkyl, C₂-C10 alkenyl, C₂-C₁₀ alkynyl, C₃-C₁₀ cycloalkyl, C₁-C10 thioalkyl, C₁-C10 hydroxylalkyl, C₁-C10 alkylaryl, and C₅-C12 aryl, C₃-C10 heterocycloalkyl, heteroaryl, —(CH₂)₃NHC(═NH)NH₂, (1H-indol-3-yl) methyl, (1H-imidazol-4-yl) methyl, and a side chain selected from a proteinogenic or non-proteinogenic amino acid; wherein the aryl groups are optionally substituted with a group selected from hydroxyl, C₁-C10 alkyl, C₁-C6 alkoxy, halogen, nitro, and cyano; or
R₉ and R₁₀ form, together with the phosphorus atoms to which they are attached, a 6-membered cycle, wherein —R₉-R₁₀— is —CH₂—CH₂—CHR—; wherein R is selected from H, a (C₅-C₆) aryl and a (C₅-C₆) heteroaryl group, wherein the aryl or heteroaryl groups are optionally substituted by a halogen, trifluoromethyl, a C₁-C6 alkyl, a C₁-C6 alkoxy, and cyano; or

R₉ and R₁₀ form, together with the phosphorus atoms to which they are attached, a 6-membered cycle, wherein —R₉-R₁₀— is —O—CH₂—CH₂—CHR—O—; wherein R is selected from H, a (C₅-C₆) aryl and a (C₅-C₆) heteroaryl group, wherein the aryl or heteroaryl groups are optionally substituted by a halogen, trifluoromethyl, a (C₁-C₆) alkyl, a (C₁-C₆) alkoxy, and cyano;
R₈ is selected from H, OR, NHR₁₃, NR₁₃R₁₄, NH—NHR₁₃, SH, CN, N₃, and halogen; wherein R₁₃ and R₁₄ selected, independently of one another, from H, (C₁-C₈) alkyl and (C₁-C₈) alkylaryl, and —CR_{B}R_{C}—C(O)—OR_{D}; wherein R_{B} and R_{C} are independently a hydrogen atom, (C₁-C₆) alkyl, (C₁-C₆) alkoxy, benzyl, indolyl or imidazolyl; wherein the (C₁-C₆) alkyl and the (C₁-C₆) alkoxy may, optionally and independently of one another, be substituted by one or more halogen, amino, amido, guanidyl, hydroxyl, thiol, or carboxyl groups; and the benzyl group is optionally substituted by one or more halogen or hydroxyl groups; or R_{B} and R_{C}, together with the carbon atom to which they are attached, form a C₃-C₆ cycloalkyl group optionally substituted with one or more halogen, amino, amido, guanidyl, hydroxyl, thiol, and carboxyl; and R_{D} is hydrogen, (C₁-C₆) alkyl, (C₂-C₆) alkenyl, (C₂-C₆) alkynyl, or (C₃-C₆) cycloalkyl;
Y is selected from CH, CH₂, C(CH₃)₂, and CCH₃;
[symbol] is a single or double bond depending on Y; and
[symbol] is the alpha or beta anomer depending on the position of R₁
   or
   the compound of formula (Ia):

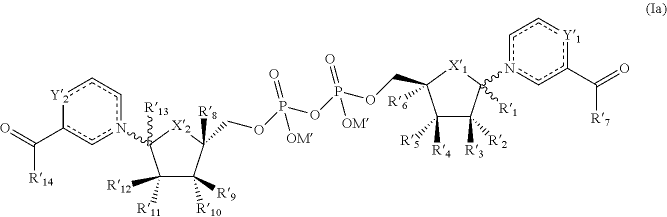

or a pharmaceutically acceptable stereoisomer, salt, hydrate, solvate, or crystal thereof, or a combination thereof, wherein
X′₁ and X′₂ are independently selected from O, CH₂, S, Se, CHF, CF₂, and C═CH₂;
R′₁ and R′₁₃ are independently selected from H, azido, cyano, C1-C8 alkyl, C1-C8 thioalkyl, C1-C8 heteroalkyl, and OR; wherein R is selected from H and C₁-C8 alkyl;
R′₂, R′₃, R′₄, R′₅, R′₉, R′₁₀, R′₁₁, R′₁₂ are independently selected from H, halogen, azido, cyano, hydroxyl, C₁-C₁₂ alkyl, C₁-C₁₂ thioalkyl, C₁-C₁₂ heteroalkyl, C₁-C₁₂ haloalkyl, and OR; wherein R may be selected from H, C₁-C₁₂ alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)(C₁-C₁₂) aryl, C(O)NH(C₁-C₁₂) alkylaryl, C(O)O(C₁-C₁₂) alkylaryl, and a C(O)CHR_{AA}NH₂ group; wherein R_{AA} is a side chain selected from the proteinogenic amino acids;
R′₆ and R′₈ are independently selected from H, azido, cyano, C₁-C₈ alkyl, and OR, wherein R is selected from H and C₁-C₈ alkyl;
R′₇ and R′₁₄ are independently selected from H, OR, NHR, NRR′, NH—NHR, SH, CN, N₃, and halogen; wherein R and R′ are independently selected from H and (C₁-C₈) alkylaryl;
Y′1 and Y′2 are independently selected from CH, CH², C(CH₃)₂ and CCH₃;
M′ is selected from H and a suitable counterion;
[symbol] is a single or double bond, depending on Y′1 and Y′2; and
[symbol] is an alpha or beta anomer depending on the position of R′₁ and R′₁₃;
   and combinations thereof, for use in the prevention and/or treatment of ankylosing spondylitis.

In a first preferred embodiment, the pharmaceutically acceptable derivative is the compound of formula (I).

In one variant of the first embodiment, X is oxygen.

In one variant of the first embodiment, R₁ and R₆ each, independently of one another, are hydrogen.

In one variant of the first embodiment, R₂, R₃, R₄, and R₅ each, independently of one another, are hydrogen or OH.

In one variant of the first embodiment, Y is CH.

In one variant of the first embodiment, Y is CHs.

In one variant of the first embodiment, R₇ is hydrogen.

In one variant of the first embodiment, R₇ is P(O)(OH)₂.

In one variant of the first embodiment, the compound of the invention is selected from compounds of formula I-A-I-J:

TABLE 1

| Compounds | |
|---|---|
| (anomers) | Structure |
| I-A (beta) | 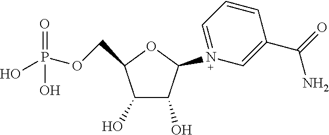 |
| I-B (alpha) | 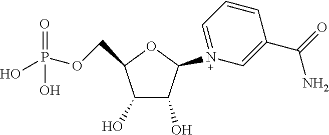 |
| I-C (beta) | 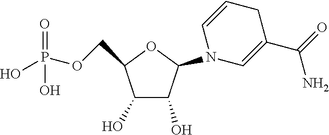 [SMILES: NC(=O)C1=CN([C@@H]2O[C@H](COP(=O)(O)O)[C@@H](O)[C@H]2O)C=CC1] |
| I-D (alpha) | 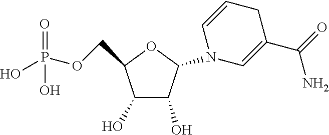 [SMILES: NC(=O)C1=CN([C@H]2O[C@H](COP(=O)(O)O)[C@@H](O)[C@H]2O)C=CC1] |
| I-E (beta) | 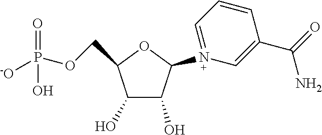 |
| I-F (alpha) | 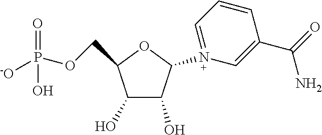 |
| I-G (beta) | 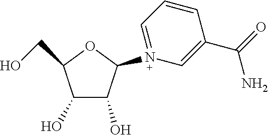 |
| I-H (alpha) | 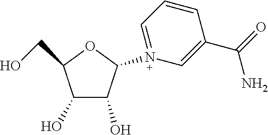 |
| I-I (beta) | 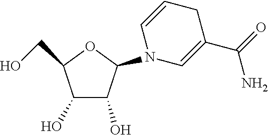 [SMILES: NC(=O)C1=CN([C@@H]2O[C@H](CO)[C@@H](O)[C@H]2O)C=CC1] |
| I-J (alpha) | 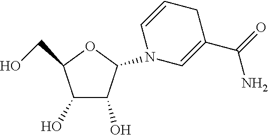 [SMILES: NC(=O)C1=CN([C@H]2O[C@H](CO)[C@@H](O)[C@H]2O)C=CC1] |

Preferably, the compound of formula (I) is selected from compound I-A, compound I-B, compound I-C, compound I-D, compound I-E, compound I-F, compound I-G, compound I-H, compound I-I, compound I-J, preferably compound I-C, compound I-D, or compound I-F, and combinations thereof. More preferably, the compound of formula (I) is selected from compound IB, compound IC, compound ID, compound IF, and combinations thereof.

In a second preferred embodiment, the pharmaceutically acceptable derivative is the compound of formula (Ia).

In one variant of the second embodiment, X′1 and X′2 are each independently oxygen.

In one variant of the second embodiment, R′7 and R′14 are each independently NH2.

In one variant of the second embodiment, R′1 and/or R′13 are each independently hydrogen.

In one variant of the second embodiment, R′6 and/or R′8 are each independently hydrogen.

In one variant of the second embodiment, R′2, R′3, R′4, R′5. R′9, R′10, R′11, and R′12 are each independently hydrogen.

In one variant of the second embodiment, R′2, R′3, R′4, R′5, R′9, R′10, R′11, and R′12 are each independently OH.

In one variant of the second embodiment, Y′1 and Y′2 are each independently CH.

In one variant of the second embodiment, Y′1 and Y′2 are each independently CH2.

In one variant of the first embodiment, the compound of the invention is selected from compounds of formula Ia-A-Ia-I:

TABLE 2

| Compounds | |
|---|---|
| (anomers) | Structure |
| Ia-A (beta, beta) | 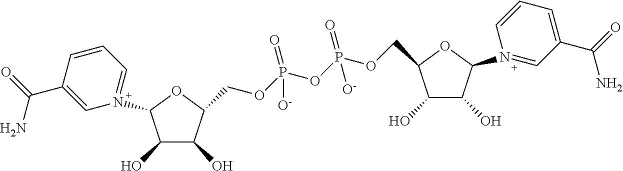 |
| Ia-B (beta, alpha) | |
| Ia-C (alpha, alpha) | |
| Ia-D (beta, beta) | |
| Ia-E (beta, alpha) | |
| Ia-F (alpha, alpha) | |
| Ia-G (beta, beta) | 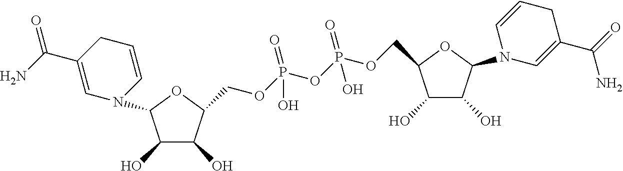 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | |
| Ia-I (alpha, alpha) | 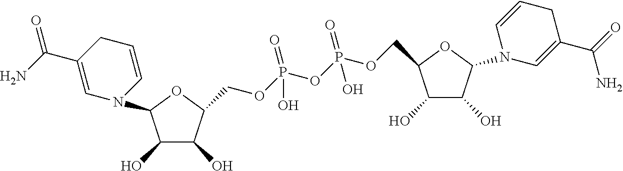 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

Preferably, the compound of formula (I) is selected from compound I-A, compound I-B, compound I-C, compound I-D, compound I-E, compound I-F, compound I-G, compound I-H, compound I-I, compound I-J, preferably compound I-C, compound I-D, or compound I-F, and combinations thereof. More preferably, the compound of formula (I) is selected from compound IB, compound IC, compound ID, compound IF, and combinations thereof.

Preferably, the compound of formula (Ia) is selected from the compounds of formula Ia-A-Ia-I, more preferably from the compound of formula Ia-B, the compound of formula Ia-C, the compound of formula Ia-E, the compound of formula Ia-F, the compound of formula Ia-H, the compound of formula Ia-I, and the compound of formula Ia-G, as well as combinations thereof.

Mode of Administration and Galenic Form

The compounds of formula (I) or (Ia), as well as the compositions according to the invention, may be administered orally, intraocularly, sublingually, intravenously, intramuscularly, intraarticularly, subcutaneously, transcutaneously, vaginally, peridurally, intravesically, rectally, or by inhalation.

Depending on the intended mode of administration, the compositions according to the invention may be provided in the form of a tablet, capsule, sachet, granulate, soft capsule, lyophilisate, suspension, gel, syrup, solution, water-in-oil emulsion, oil-in-water emulsion, oil, cream, milk, spray, ointment, ampule, suppository, eye drops, vaginal ovule, vaginal capsule, liquid for inhalation, dry powder inhaler, pressurised metered-dose inhaler.

In a preferred embodiment, the compounds of formula (I) or (Ia), as well as the compositions according to the invention are administered by injection, in particular subcutaneously, intravenously, or intraarticularly, preferably intraarticularly.

In a preferred embodiment, the compounds of formula (I) or (Ia), as well as the compositions according to the invention are administered orally.

The oral form according to the invention may also be an immediate release form: such a galenic form allows for rapid absorption of the NAD precursor and reduced delay in onset of action. Galenic forms for immediate release include, inter alia, dispersible, effervescent, orodispersible, and sublingual tablets.

Dispersible tablets are uncoated or film-coated tablets that may be dispersed in a liquid prior to administration in order to ensure homogeneous dispersion. The dispersible tablets usually disintegrate within three minutes once placed in water or another liquid.

An effervescent tablet is a tablet designed to fragment and dissolve rapidly in water or another liquid whilst releasing carbon dioxide (CO2). This release causes effervescence and the fragmentation of the tablet.

An orodispersible tablet is a dispersible tablet that is placed on the tongue. The active ingredient is then absorbed by the gastrointestinal mucosa.

‘Sublingual tablet’ refers to an oral lyophilisate that is placed under the tongue so that the active ingredient is absorbed by the sublingual mucosa, in particular by the lingual vein and artery.

The oral form according to the invention may also be a delayed release form: The NAD precursor is dissolved and absorbed in the intestines, thus limiting gastric irritation or the breakdown of fragile active ingredients at acidic pH. These are mostly gastroresistant forms, i.e. the tablets or granulates are coated in a polymer film that is insoluble in an acidic medium but permeable to water in an alkaline medium, or a lipid film that is broken down by the intestinal lipases.

‘Gastroresistant’ refers to a galenic form that does not dissolve in the stomach. Such galenic forms are for delayed release, i.e. they have a coating or a coating composition that is resistant to the acidic pH of the stomach (pH<2) in order to dissolve in the intestines. Whether a galenic form is gastroresistant is determined based on the test established by the European Pharmacopoeia. In brief, the gastroresistance of a capsule is measured in a disintegration medium of 0.1 M hydrochloric acid at 37° C. in a disintegration device. This environment mimics the physico-chemical conditions of the stomach. The capsules are incubated in this environment for 1 h. The capsule must not show any signs of disintegration or fissures that might result in loss of contents. Then, the capsule is incubated for 1 h in a phosphate buffer solution at pH 6.8 at 37° C.; this solution mimics the conditions of the intestinal environment in accordance with the recommendations of the European Pharmacopoeia. The capsule must be totally disintegrated within less than one hour.

The oral form according to the invention may also be a sustained and sequential release form: Sequential (release at precise time intervals) and sustained release forms (continuous release of active ingredient until exhaustion) spread out the release of the active ingredient over time in order to maintain an effective plasma concentration for a longer time in the patient's body. Such galenic forms allow for pain relief over a longer period of time, and make it possible to increase the interval between doses of the drug.

In a more preferred embodiment, the compounds of formula (I) or (Ia), as well as the composition according to the invention are administered orally in the form of a gastroresistant capsule or a sublingual tablet. These galenic forms allow for better absorption and better distribution to all organs.

The mode of administration and galenic form are determined by persons skilled in the art depending on the anatomic site of the pain to be treated and the patient.

Therapeutic Combinations

The compounds of formula (I) or (Ia), as well as the compositions according to the invention may also be used in combination with at least one additional therapeutic agent, in particular therapeutics agent commonly used to treat AS eruptions.

More specifically, analgesics, NSAIDs, cortisone, and cortisone derivatives may be used to treat and relieve AS eruptions.

The analgesic may be selected from paracetamol, aspirin, codeine, dihydrocodeine, tramadol, morphine, buprenorphin, fentanyl, hydromorphone, nalbuphine, oxycodone, pethidine, and combinations thereof.

Preferably, the analgesic is an NSAID. The NSAID may be selected from ibuprofen, ketoprofen, naproxen, alminoprofen, aceclofenac, mefenamic acid, niflumic acid, tiaprofenic acid, celecoxib, dexketoprofen, diclofenac, etodolac, etoricoxib, fenoprofen, flurbiprofen, indomethacin, meloxicam, nabumetone, piroxicam, sulindac, tenoxicam, and combinations thereof. The combined administration of an NSAID and NMN is of particular interest for the treatment of ankylosing spondylitis. On the one hand, the administration of NMN in combination with NSAIDs allows for a reduced frequency of NSAID administration, as well as a reduction in the NSAID dose administered. Thus, it allows for a reduction in the side-effects of NSAIDs for the patient. This also allows for the efficacy of NSAIDs to be extended in patients before their treatment is modified.

The cortisone derivative may be selected from betamethasone, ciprofloxacin, cortivazol, dexamethasone, fludrocortisone, methylprednisolone, prednisolone, triamcinolone, and combinations thereof.

The compounds of formula (I) or (Ia), as well as the compositions according to the invention, may also be administered in combination with a treatment for AS such as an immunosuppressant, immunomodulator, an anti-TNF agent, an anti-interleukin, or combinations thereof. The use of such a treatment in combination with NMN or a derivative or salt thereof, as well as compositions comprising them is also compatible with the administration of analgesics, NSAIDs, cortisone, and/or cortisone derivatives in order to treat eruptions.

‘Anti-TNF agent’ refers to molecules capable of inhibiting the action of TNF, such as anti-TNF antibodies, anti-TNF receptor antibodies, or competitive or non-competitive TNF receptor binding inhibitors. Likewise, ‘anti-interleukin’ refers to molecules capable of inhibiting the action of a specific interleukin, such as antibodies against that interleukin, antibodies against the receptor of that interleukin, or competitive or non-competitive inhibitors of the binding of that interleukin to its receptor.

The immunosuppressant may be selected from azathioprine, cyclophosphamide, chlorambucil, cyclosporine, methotrexate, and combinations thereof. Preferably, the immunosuppressant may be methotrexate or cyclosporine, more preferably methotrexate.

The immunomodulator may be selected from leflunomide, sulphasalazine, and combinations thereof, preferably sulphasalazine.

The anti-TNF agent may be selected from infliximab, etanercept, adalimumab, certolizumab, golimumab, and combinations thereof. The combined use of an anti-TNF agent and NMN in the treatment of AS may be of particular interest, whether the AS in question is axial or peripheral.

The anti-interleukin may be an interleukin 17 inhibitor selected from ixekizumab and secukinumab. The anti-interleukin may also be an anti-interleukin 12, preferably ustekinumab. Ustekinumab is an antibody capable of targeting interleukin 12 and interleukin 23.

In particular, the administration of the compounds of formula (I) or (Ia) according to the invention in association with one of the aforementioned AS treatments allows for a reduction in the frequency of administration of these treatments and/or in the dose administered, and thus in the incidence and severity of the adverse effects associated with these treatments. The combined administration of the compounds according to the invention with conventional AS treatments also allows for the time at which these treatments lose their efficacy against the disease to be delayed. The combined administration of the compounds according to the invention with these treatments thus allows for their efficacy to be extended, and thus to give patients the benefit of therapeutic alternatives.

Compositions

The composition according to the invention comprises a compound of formula (I) and/or a compound of formula (Ia) and at least one pharmaceutically acceptable excipient. The composition according to the invention may further comprise at least one additional therapeutic agent such as those mentioned supra for use in the prevention and/or treatment of AS as described supra.

In the context of this invention, ‘excipient’ refers to any substance other than the compounds according to the invention or a therapeutic agent in the composition that has no therapeutic effect. The excipient does not interact chemically with NMN or any additional therapeutic agent.

The excipient may be selected from bulking agents, lubricants, flavourings, colourings, emulsifiers, compacting agents, gellants, plasticisers, surfactants, or combinations thereof. Persons skilled in the art will know which excipients to select based on the galenic form selected.

The composition according to the invention may be a pharmaceutical composition. In this case, the excipient is a pharmaceutically acceptable excipient as defined supra.

The composition according to the invention may also be a dietary supplement.

Another object of the invention is a kit comprising a compound of formula (I) as defined supra, and/or a compound of formula (Ia) as defined supra, and/or a composition according to the invention as defined supra and at least one additional therapeutic agent for use in the prevention and/or treatment of AS as described supra.

Method for Preparing Compounds of Formula (I) and (Ia)

The compounds of formula (I) or (Ia) may be prepared by any method well known to persons skilled in the art.

Method for Preparing Compounds of Formula (I)

The compounds of formula (I) may be prepared according to the method described in international application WO 2017/024255A1, as well as the method described infra.

In particular, the compounds of formula (I) disclosed herein may be prepared as described infra from the substrates A-E. It will be understood by persons skilled in the art that these reaction schemes are by no means limiting, and that variations are possible without departing from the spirit and scope of this invention.

In one embodiment, the invention concerns a method for preparing compounds of formula (I) as described supra.

In a first step, the method includes monophosphorylating a compound of formula (A) in the presence of phosphoryl chloride and a trialkyl phosphate in order to afford the phosphorodichloridate of formula (B),

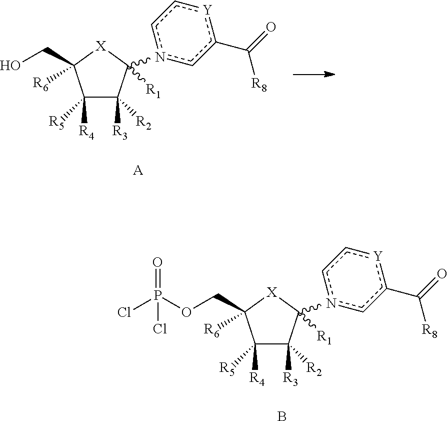

wherein X, R₁, R₂, R₃, R₄, R₅, R₆, R₈, Y, [symbol] , and [symbol] are as defined supra for compounds of formula (I).

In a second step, the phosphorodichloridate of formula (B) is hydrolysed, resulting in the phosphate of formula (C),

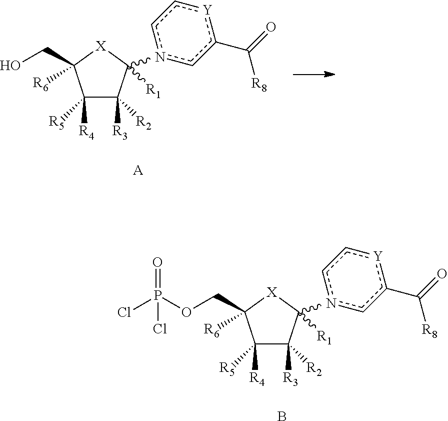

wherein X, R₁, R₂, R₃, R₄, R₅, R₆, R₈, Y, [symbol] , and [symbol] are as defined supra for compounds of formula (I).

In one embodiment, the compound of formula (A) is synthesised using various methods known to persons skilled in the art.

In one embodiment, the compound of formula (A) is synthesised by reacting the pentose of formula (D) with a nitrogenous derivative of formula (E), wherein R, R₂, R₃, R₄, R₅, R₆, R₇, Y are as described supra for the compounds of formula I, resulting in the compound of formula (A-1), which is then selectively deprotected to afford the compound of formula (A),

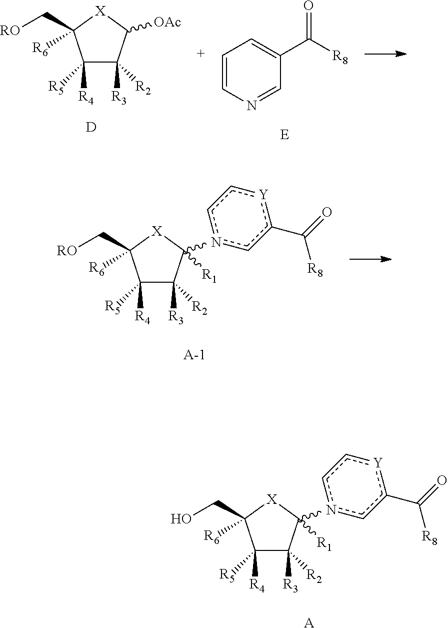

wherein X, R₁, R₂, R₃, R₄, R₅, R₆, R₈, Y, [symbol] , and [symbol] are as defined supra for compounds of formula (I).

In one embodiment, R is a suitable protecting group known to persons skilled in the art. In one embodiment, the protecting group is selected from triarylmethyls and/or silyls. Non-limiting examples of triarylmethyl include trityl, monomethoxytrityl, 4,4′-dimethoxytrityl, and 4,4′,4″-trimethoxytrityl groups. Non-limiting examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl groups.

In one embodiment, any hydroxyl group attached to the pentose is protected by a suitable protecting group known to persons skilled in the art.

The selection and exchange of protecting groups is within the general knowledge of persons skilled in the art. Protecting groups may also be eliminated by methods well known to persons skilled in the art, e.g. with an acid (e.g. a mineral or organic acid), a base, or a fluoride source.

In a preferred embodiment, the nitrogenous derivative of formula (E) is coupled to the pentose of formula (D) by a reaction in the presence of a Lewis acid, resulting in the compound of formula (A-1). Non-limiting examples of Lewis acids include TMSOTf, BF₃.OEt₂, TiCl₄, and FeCl₃.

In one embodiment, the method of this invention further comprises a step of reducing the compound of formula (A) by various methods well known to persons skilled in the art, resulting in the compound of formula (A′), wherein [sic] is CH₂, and R₁, R₂, R₃, R₄, R₅, R₆, R₈, Y, [symbol] , and [symbol] are as defined supra for compounds of formula (I).

In one particular embodiment, this invention concerns a method for preparing compounds of formula I-A, I-C, I-E, I-G.

In a first step, the nicotinamide of formula E is coupled to the ribose tetraacetate of formula D by a reaction in the presence of a Lewis acid, resulting in the compound of formula A-1.

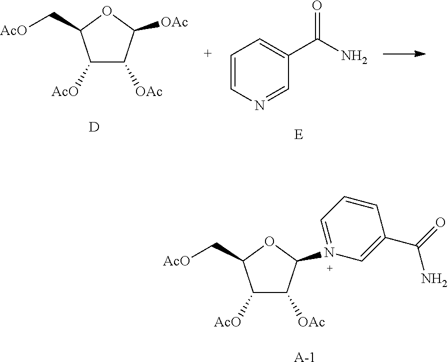

In a second step, the compound of formula A-1 is treated with ammonia, resulting in the compound of formula I-A:

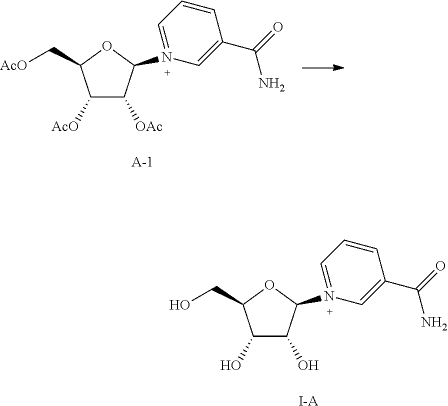

In a third step, monophosphorylation of the compound of formula I-A in the presence of phosphoryl chloride and a trialkyl phosphate results in the phosphorodichloridate of formula I-A′:

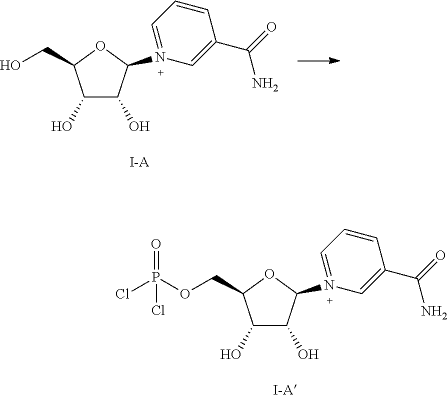

In a fourth step, the phosphorodichloridate of formula B is hydrolysed, resulting in the compound of formula I-C,

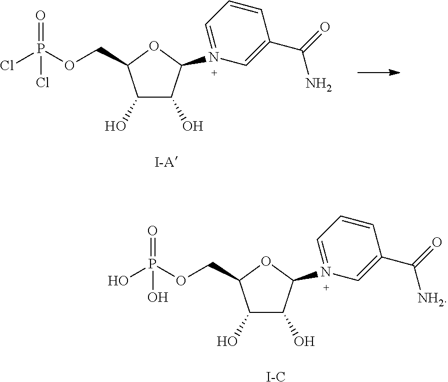

In one embodiment, a step of reducing the compound of formula I-A is carried out, resulting in the compound of formula I-E:

The compound of formula I-E is then monophosphorylated, as described for the fourth step, and hydrolysed, resulting in the compound of formula I-G.

In one embodiment, the compounds of formula (I) are selected from the compounds I-A-I-J in the following table:

TABLE 1

| Compounds | |
|---|---|
| (anomers) | Structure |
| I-A (beta) | 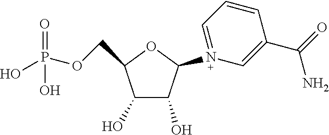 |
| I-B (alpha) | 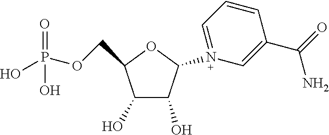 |
| I-C (beta) | 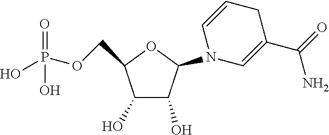 [SMILES: NC(=O)C1=CN([C@@H]2O[C@H](COP(=O)(O)O)[C@@H](O)[C@H]2O)C=CC1] |
| I-D (alpha) | 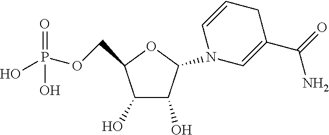 [SMILES: NC(=O)C1=CN([C@H]2O[C@H](COP(=O)(O)O)[C@@H](O)[C@H]2O)C=CC1] |
| I-E (beta) | 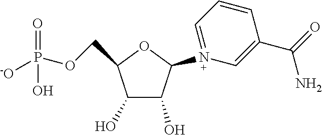 |
| I-F (alpha) | 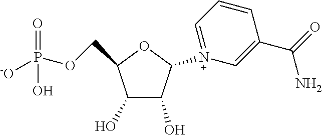 |
| I-G (beta) | 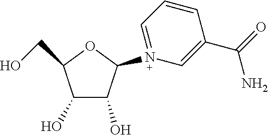 |
| I-H (alpha) | 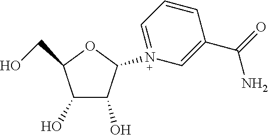 |
| I-I (beta) | 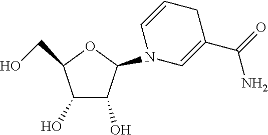 [SMILES: NC(=O)C1=CN([C@@H]2O[C@H](CO)[C@@H](O)[C@H]2O)C=CC1] |
| I-J (alpha) | 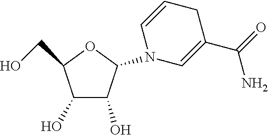 [SMILES: NC(=O)C1=CN([C@H]2O[C@H](CO)[C@@H](O)[C@H]2O)C=CC1] |

Preferably, the compound of formula (I) is selected from compound I-A, compound I-B, compound I-C, compound I-D, compound I-E, compound I-F, compound I-G, compound I-H, compound I-I, compound I-J, preferably compound I-C, compound I-D, or compound I-F, and combinations thereof. More preferably, the compound of formula (I) is selected from compound IB, compound IC, compound ID, compound IF, and combinations thereof.

Method for Preparing Compounds of Formula (Ia)

In particular, the compounds of formula (I) presented herein may be prepared as described infra from the substrates X-XIII. It will be understood by persons of ordinary skill in the art that these schemes are by no means limiting, and that variations of individual details are possible without departing from the spirit and scope of this invention.

In one embodiment, the invention concerns a method for preparing the compound of formula I as described supra.

The method consists first of monophosphorylating a compound of formula X in the presence of phosphoryl chloride in a trialkyl phosphate, in order to obtain the phosphorodichloridate compound XI,

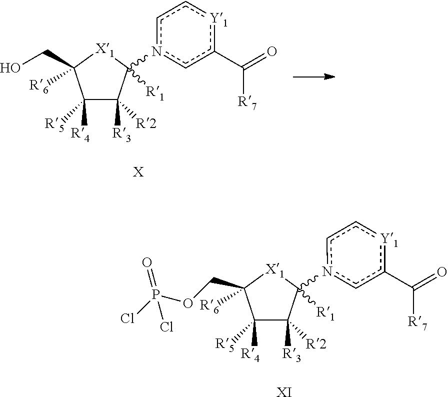

wherein X′₁, R′₁, R′₂, R′₃, R′₄, R′₅, R′₆, R′₇, Y′₁, [symbol] , and [symbol] are as defined supra.

In a second step, hydrolysing the phosphorodichloridate XI obtained in the first step gives the phosphate compound of formula XII,

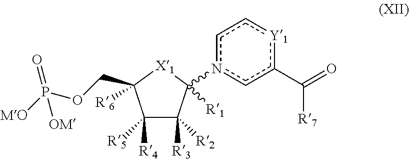

wherein X′₁, R′₁, R′₂, R′₃, R′₄, R′₅, R′₆, R′₇, Y′₁, M′, [symbol] , and [symbol] are as defined supra.

The phosphate compound of formula XII obtained in the second step is then reacted with a phosphorodichloridate compound of formula XIII that was obtained as described in the first step,

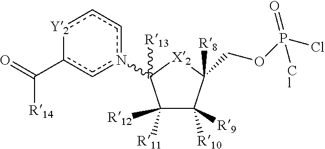

wherein X′₈, R′₂, R′₂, R′₁₀, R′₁₁, R′₁₂, R′₁₃, R′₁₄, Y′₁, [symbol] , and [symbol] are as described for formula Ia. to give the compound of formula Ia as described herein.

In one embodiment, the method further comprises a step of reducing the compound of formula Ia using various methods known to specialists to give the compound of formula Ia, wherein Y′₁ and Y′₂ are identical and are each CH₂. and wherein X′₁, X′₂, R′₁, R′₂, R′₃, R′₄, R′₅, R′₆, R′₇, R′₈, R′₉, R′₁₀, R′₁₁, R′₁₂, R′₁₃, R′₁₄, Y′₁, Y′₂, and [symbol] are as described herein for formula Ia.

In one variant, R is a suitable protecting group known to persons skilled in the art. Examples of suitable protecting groups include triarylmethyl and/or silyl groups. Non-limiting examples of triarylmethyl include trityl, monomethoxytrityl, 4,4′-dimethoxytrityl, and 4,4′,4″-trimethoxytrityl. Non-limiting examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl.

In one representation, any hydroxy group attached to the pentose ring is protected by a suitable protecting group known to persons skilled in the art.

The selection and exchange of protecting groups fall within the general knowledge of persons skilled in the art. Any protecting group may also be eliminated by methods known to persons skilled in the art, e.g. with an acid (e.g. a mineral or organic acid), a base, or a fluoride source.

In one preferred embodiment, nitrogenous derivatives of formula XV are added to the pentose XIV for a coupling reaction in the presence of a Lewis acid to give the compound of formula X-1. Non-limiting examples of suitable Lewis acids include TMSOTf, BF3.OEt2, TiCl4, and FeCl3.

In one specific embodiment, the invention concerns a method for preparing the compound of formula VIII,

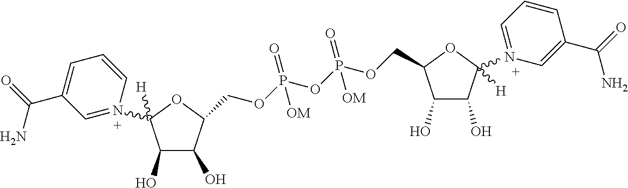

or pharmaceutically acceptable salts and/or solvates thereof.

In a first step, the nicotinamide of formula XV is coupled to the ribose tetraacetate XIV by a coupling reaction in the presence of a Lewis acid, to give in the compound of formula X-1.

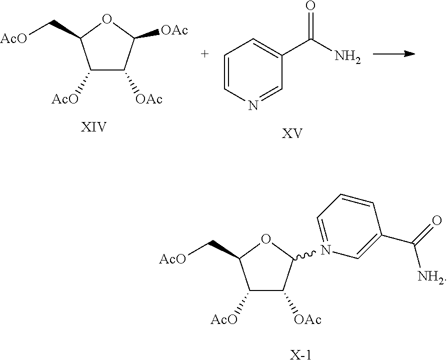

In a second step, treating the compound of formula X-1 with ammonia affords the compound of formula X:

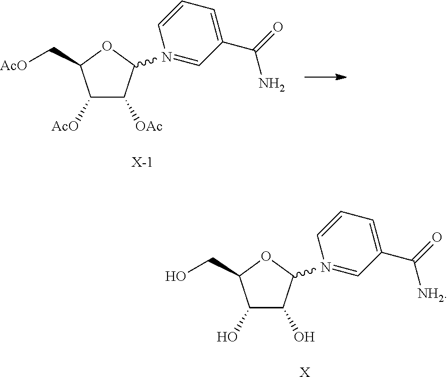

In a third step, monophosphorylating a compound of formula X in the presence of phosphoryl chloride in a trialkyl phosphate affords the phosphorodichloridate compound XI:

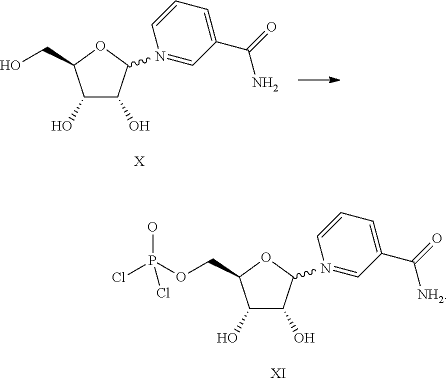

In a fourth step, the phosphorodichloridate compound XI obtained in the third step is partially hydrolysed to afford the phosphate compound of formula XII:

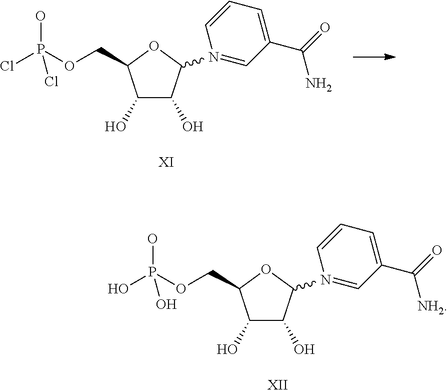

In a fifth step, the phosphate compound of formula XII obtained in the fourth step is reacted with the phosphorodichloridate compound of formula XI obtained as described in the third step, to obtain the compound of formula VIII.

In another specific embodiment, the invention concerns a method for preparing the compound of formula IX,

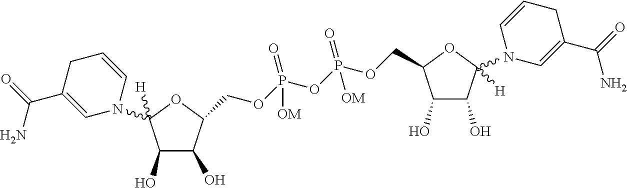

or pharmaceutically acceptable salts and/or solvates thereof.

In one variant, the compound of formula IX is obtained from the compound of formula VIII, previously synthesised as described supra.

In this embodiment, the compound of formula IX is obtained by reducing the compound of formula VIII using a suitable reducing agent known to persons skilled in the art to afford the compound of formula IX.

In one embodiment, preferred compounds of the invention are the compounds Ia-A-Ia-I of table 2:

TABLE 2

| Compounds | |
|---|---|
| (anomers) | Structure |
| Ia-A (beta, beta) | 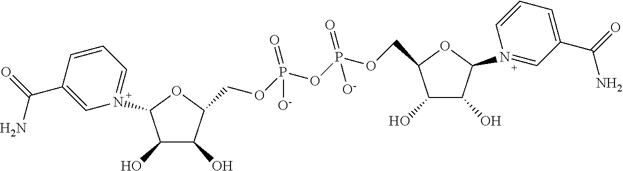 |
| Ia-B (beta, alpha) | |
| Ia-C (alpha, alpha) | |
| Ia-D (beta, beta) | |
| Ia-E (beta, alpha) | |
| Ia-F (alpha, alpha) | |
| Ia-G (beta, beta) | 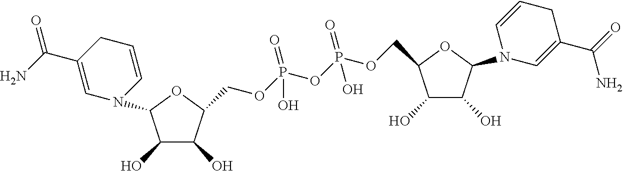 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | |
| Ia-I (alpha, alpha) | 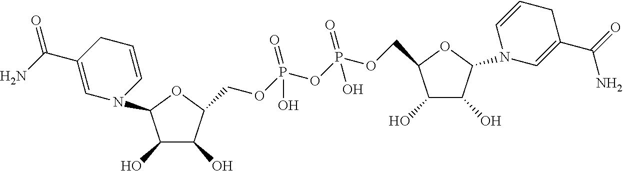 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

Preferably, the compound of formula (Ia) is selected from the compound of formula Ia-B, the compound of formula Ia-C, the compound of formula Ia-E, the compound of formula Ia-F, the compound of formula Ia-H, the compound of formula Ia-I, and the compound of formula Ia-G, as well as combinations thereof.

### FIGURES

FIG. 1 is a graph of the development of the average weight of mice as a function of treatments.

FIG. 2 is a graph of the development of the average joint score as a function of treatments.

FIG. 3 is a graph of the development of the average tail score as a function of treatments.

### EXAMPLE

In the following, the examples are provided solely to illustrate this invention, and by no means to limit its scope.

The efficacy of the use of NMN (compound IE) according to the invention was evaluated in 3-week-old TgA86 mice as a model for ankylosing spondylitis. TgA86 mice develop peripheral and axial AS with an incidence of 100%. In these mice, the peripheral form is manifested by joint welling and paw deformation, whilst the axial form is clinically characterised by curved tails and pelvic ankylosis.

In brief, male TgA86 mice were divided into two groups of 10 mice each: (i) a control group, in which the mice were treated with vehicle, i.e. a 0.9% NaCl solution (10 mL/kg), marked ‘Vehicle’; (iv) a group of mice treated with NMN (185 mg/kg), marked ‘NMN’. The solutions were injected intraperitoneally.

The mice were treated for 10 weeks under the conditions set forth supra. The joint score, tail score, and weight of the mice were measured every week. The joint score was determined in accordance with table 3 infra:

TABLE 3
Peripheral pathology

| Joint score | Interpretation | Parameters |
|---|---|---|
| 0 | No disease | No arthritis (normal appearance, |
| | | mouse can support its weight by |
| | | clinging to an inverted or inclined |
| | | surface such as a wire rack or a cage |
| | | cover for a certain amount of time, |
| | | maximum grip force). |
| 0.5 | Mild disease | Appearance of arthritis: Mild joint |
| | | swelling, all other parameters as with |
| | | a score of 0. |
| 1 | Mild-to-moderate | Joint deformation due to swelling, |
| | disease | paw inflammation, all other |
| | | parameters as with a score of 1. |
| 1.5 | Moderate disease | Paw joint swelling, deformation + |
| | | inward deformation of the last digit, |
| | | brief support when mouse clings to an |
| | | inverted or inclined surface such as a |
| | | wire rack or a cage cover, reduced |
| | | grip force. |
| 2 | Moderate-to-severe | Significant swelling of paw joint and |
| | disease | digits, deformation of leg joint, |
| | | deformation, no support when clinging |
| | | to an inverted or inclined surface such |
| | | as a wire rack or a cage cover, no grip |
| | | force, climbing/feeding affected. |

The tail score is an indicator that allows for an evaluation of the axial form of AS and pelvic ankylosis (see https://www.biomedcode.com/wp-content/uploads/2019/07/TgA86-white-paper.pdf). The tail score was determined in accordance with table 4 infra:

TABLE 4
Axial pathology

| Tail score | Interpretation | Parameters |
|---|---|---|
| 0 | No disease | Normal. Normal tail phenotype. |
| 1 | Moderate disease | Normal tail with first signs of tail flexion |
| | | (mild flexion in one or more places). |
| 2 | Moderate-to-severe | Multiple mild-to-severe tail flexions |
| | disease | (tight flexions). |
| 3 | Severe disease | Tail ankylosis with severe, tight tail |
| | | flexions. |

The significance of the values analysed with the Student test (T-test) are indicated as indicated in table 5:

TABLE 5

| | Symbol |
|---|---|
| T-test > 0.1 | * |
| T-test > 0.05 | ** |

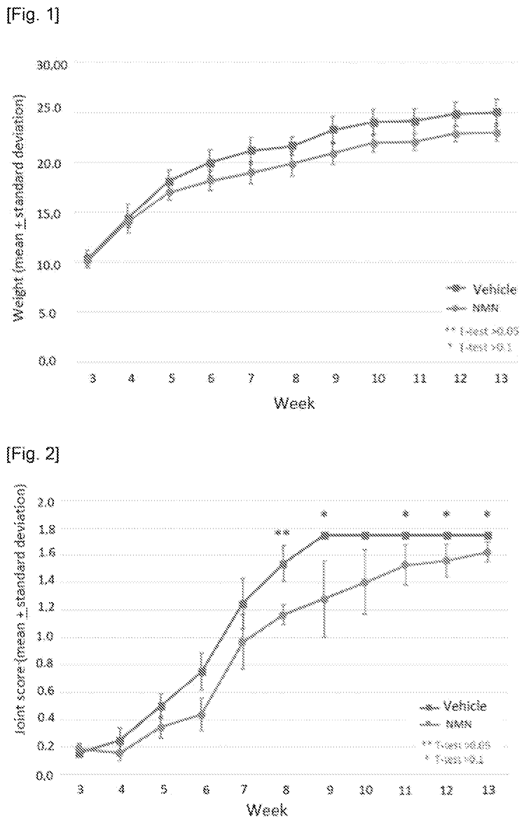

As can be seen in FIG. 1, the weight of the mice treated with NMN follows a similar course of development to that of the control group. The decrease observed is not significant. This result shows that NMN is well tolerated by the mice and does not cause any distress or toxicity in the animals. Thus, the administration of NMN is safe.

As can be seen in FIG. 2, the administration of NMN allows for a reduction in the joint score of the mice compared to the control group, i.e. the administration of NMN reduces joint inflammation in this model of peripheral AS. Moreover, the reduction is particularly significant starting in the 5^{th} week of treatment, thus showing the efficacy of treatment with NMN for joint swelling induced by AS.

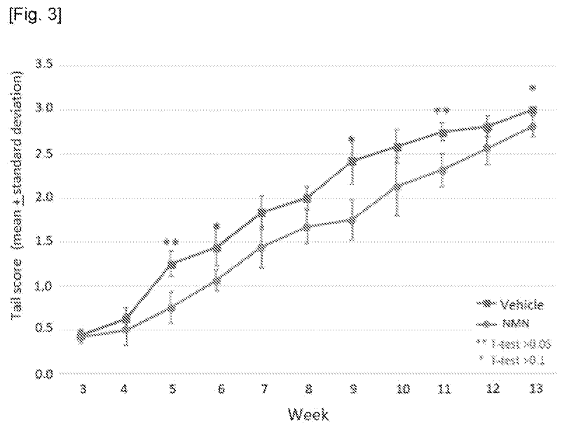

FIG. 3 shows the development of the tail score, which allows for a determination of whether the axial form of AS is improving or worsening in this mouse model. As can be seen, the administration of NMN reduces the tail score in the mice treated compared to the control group; i.e. the spinal column of the mice is less deformed and the pelvis less ankylosed. The reduction is significant starting in the second week of treatment. As such, NMN is effective to treat the axial form of AS.

The compounds of formula (I) or (Ia), as well as compositions comprising them, can thus be used safely and successfully to treat and prevent ankylosing spondylitis. Thus, this invention provides a therapeutic alternative to conventional treatments for AS, or at least proposes a therapeutic adjunct to conventional treatments in order to reduce their frequency and dosage. Given the safety of the compounds according to the invention, as well as the compositions comprising them, this invention is capable of treating and/or preventing AS without inducing the side-effects caused by conventional treatments.

1. Compound of the formula (I): 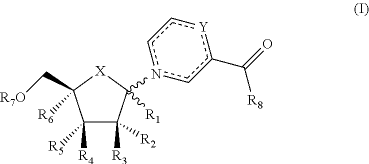
or a pharmaceutically acceptable stereoisomer, salt, hydrate, solvate, or crystal thereof, wherein
X is selected from O, CH₂, S, Se, CHF, CF₂, C═CH₂;
R₁ is selected from H, azido, cyano, C₁-C8 alkyl, C₁-C8 thioalkyl, C₁-C8 heteroalkyl, and OR; wherein R is selected from H and C₁-C8 alkyl;
R₂, R₃, R₄, and R₅ are selected independently of one another from H, halogen, azido, cyano, hydroxyl, C₁-C12 alkyl, C₁-C12 thioalkyl, C₁-C12 heteroalkyl, C₁-C12 haloalkyl, and OR; wherein R is selected from H, C₁-C₁₂ alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)NH(C₁-C₁₂) alkylaryl, C(O)O(C₁-C₁₂) alkylaryl, and C(O)CHR_{AA}NH₂; wherein R_{AA} is a side chain selected from the proteinogenic amino acids;
R₆ is selected from H, azido, cyano, C₁-C8 alkyl, C₁-C8 thioalkyl, C₁-C8 heteroalkyl, and OR; wherein R is selected from H and C₁-C8 alkyl;
R₇ is selected from H, P(O)R₉R₁₀, and P(S)R₉R₁₀; wherein
R₉ and R₁₀ are selected independently of one another from OH, OR₁₁, NHR₁₃, NR₁₃R₁₄, C₁-C₈ alkyl, C₂-C₈ alkenyl, C₂-C₈ alkynyl, C₃-C10 cycloalkyl, C₅-C12 aryl, (C₁-C₈) alkylaryl, (C₁-C₈) arylalkyl, (C₁-C₈) heteroalkyl, (C₁-C₈) heterocycloalkyl, heteroaryl, and NHCHR_{A}R_{A′}C(O)R₁₂; wherein:
R₁₁ is selected from a C₁-C10 alkyl, C₃-C10 cycloalkyl, C₅-C18 aryl, C₁-C10 alkylaryl, substituted C₅-C12 aryl, C₁-C10 heteroalkyl, C₃-C10 heterocycloalkyl, C₁-C₁₀ haloalkyl, heteroaryl, —(CH₂)ₙC(O)(C₁-C₁₅) alkyl, —(CH₂)ₙOC(O)(C₁-C₁₅) alkyl, —(CH₂)ₙOC(O)O(C₁-C₁₅) alkyl, —(CH₂)ₙSC(O)(C₁-C₁₅) alkyl, —(CH₂)ₙC(O)O(C₁-C₁₅) alkyl, and —(CH₂)ₙC(O)O(C₁-C₁₅)alkylaryl group; wherein n is an integer from 1-8; P(O)(OH)OP(O)(OH)₂. halogen, nitro, cyano, C₁-C₆ alkoxy, C₁-C₆ haloalkoxy, —N(R₁₁ₐ)₂, C₁-C₆ acylamino, —COR_{11b}, —OCOR_{11b}; NHSO₂(C₁-C₆ alkyl), —SO₂N(R₁₁ₐ)₂SO₂; wherein each R₁₁ₐ is independently selected from H and C₁-C₆ alkyl, and R_{11b} is independently selected from OH, C₁-C₆ alkoxy, NH₂, NH(C₁-C₆ alkyl), and N(C₁-C6 alkyl)₂;
R₁₂ is selected from H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the aryl or heteroaryl groups are optionally substituted with one or two groups selected from halogen, trifluoromethyl, C₁-C₆ alkyl, C₁-C₆ alkoxy, and cyano; and
R_{A} and R_{A′} are independently selected from H, C₁-C10 alkyl, C₂-C10 alkenyl, C₂-C₁₀ alkynyl, C₃-C₁₀ cycloalkyl, C₁-C10 thioalkyl, C₁-C10 hydroxylalkyl, C₁-C10 alkylaryl, and C₅-C12 aryl, C₃-C10 heterocycloalkyl, heteroaryl, —(CH₂)₃NHC(═NH)NH₂, (1H-indol-3-yl) methyl, (1H-imidazol-4-yl) methyl, and a side chain selected from a proteinogenic or non-proteinogenic amino acid; wherein the aryl groups are optionally substituted with a group selected from hydroxyl, C₁-C10 alkyl, C₁-C6 alkoxy, halogen, nitro, and cyano; or
R₉ and R₁₀ form, together with the phosphorus atoms to which they are attached, a 6-membered cycle, wherein —R₉-R₁₀— is —CH₂—CH₂—CHR—; wherein R is selected from H, a (C₅-C₆) aryl, and a (C₅-C₆) heteroaryl group; wherein the aryl or heteroaryl groups are optionally substituted with halogen, trifluoromethyl, C₁-C₆ alkyl, C₁-C6 alkoxy, and cyano; or
R₉ and R₁₀ form, together with the phosphorus atoms to which they are attached, a 6-membered cycle, wherein —R₉-R₁₀— is —O—CH₂—CH₂—CHR—O—; wherein R is selected from H, a (C₅-C₆) aryl, and a (C₅-C₆) heteroaryl group; wherein the aryl or heteroaryl groups are optionally substituted with halogen, trifluoromethyl, (C₁-C₆) alkyl, (C₁-C6) alkoxy, and cyano;
R₈ is selected from H, OR, NHR₁₃, NR₁₃R₁₄, NH—NHR₁₃, SH, CN, N₃, and halogen; wherein R₁₃ and R₁₄ selected, independently of one another, from H, (C₁-C₈) alkyl, (C₁-C₈) alkylaryl, and —CR_{B}R_{C}—C(O)—OR_{D}; wherein R_{B} and R_{C} are independently a hydrogen atom, (C₁-C₆) alkyl, (C₁-C₆) alkoxy, benzyl, indolyl or imidazolyl; wherein the (C₁-C₆) alkyl and the (C₁-C₆) alkoxy may, optionally and independently of one another, be substituted by one or more halogen, amino, amido, guanidyl, hydroxyl, thiol, or carboxyl groups; and the benzyl group is optionally substituted by one or more halogen or hydroxyl groups; or R_{B} and R_{C}, together with the carbon atom to which they are attached, form a C₃-C₆ cycloalkyl group optionally substituted with one or more halogen, amino, amido, guanidyl, hydroxyl, thiol, and carboxyl; and R_{D} is hydrogen, (C₁-C₆) alkyl, (C₂-C₆) alkenyl, (C₂-C₆) alkynyl, or (C₃-C₆) cycloalkyl;
Y is selected from CH, CH₂, C(CH₃)₂, and CCH₃; [symbol]
is a single or double bond depending on Y; and [symbol]
is the alpha or beta anomer depending on the position of R₁
or
a compound of formula (Ia): 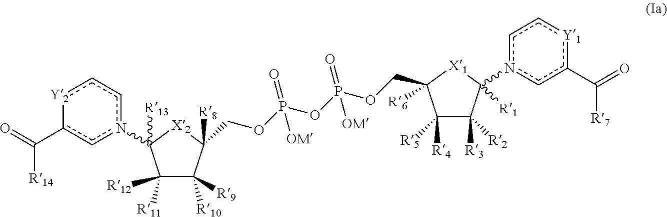
or a pharmaceutically acceptable stereoisomer, salt, hydrate, solvate, or crystal thereof, or a combination thereof, wherein
X′₁ and X′₂ are independently selected from O, CH₂, S, Se, CHF, CF₂, and C═CH₂;
R′₁ and R′13 are independently selected from H, azido, cyano, C1-C8 alkyl, C1-C8 thioalkyl, C1-C8 heteroalkyl, and OR; wherein R is selected from H and C₁-C8 alkyl;
R′₂, R′₃, R′₄, R′₅, R′₉, R′₁₀, R′₁₁, R′₁₂ are independently selected from H, halogen, azido, cyano, hydroxyl, C₁-C₁₂ alkyl, C₁-C₁₂ thioalkyl, C₁-C₁₂ heteroalkyl, C₁-C₁₂ haloalkyl, and OR; wherein R may be selected from H, C₁-C₁₂ alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)(C₁-C₁₂) aryl, C(O)NH(C₁-C₁₂) alkylaryl, C(O)O(C₁-C₁₂) alkylaryl, and a C(O)CHR_{AA}NH₂ group; wherein R_{AA} is a side chain selected from the proteinogenic amino acids;
R′₆ and R′₈ are independently selected from H, azido, cyano, C₁-C₈ alkyl, and OR, wherein R is selected from H and C₁-C₈ alkyl;
R′₇ and R′₁₄ are independently selected from H, OR, NHR, NRR′, NH—NHR, SH, CN, N₃, and halogen; wherein R and R′ are independently selected from H and (C₁-C₈) alkylaryl;
Y′1 and Y′2 are independently selected from CH, CH₂, C(CH₃)₂ and CCH₃;
M′ is selected from H and a suitable counterion; [symbol]
is a single or double bond, depending on Y′1 and Y′2; and [symbol]
is an alpha or beta anomer depending on the position of R′₁ and R′₁₃;
and combinations thereof, for use in the prevention and/or treatment of ankylosing spondylitis.

2. Compound of formula (I) for use according to claim 1, selected from compound I-A, compound I-B, compound I-C, compound I-D, compound I-E, compound I-F, compound I-G, compound I-H, compound I-I, compound I-J, preferably compound I-C, compound I-D, or compound I-F, preferably compound IB, compound IC, compound ID, compound IF, and combinations thereof, preferably from compound IB, compound IC, compound ID, compound IF, and combinations thereof.

3. Compound of formula (Ia) for use according to claim 1, selected from compounds Ia-A to Ia-I, preferably from the compound of formula Ia-B, the compound of formula Ia-C, the compound of formula Ia-E, the compound of formula Ia-F, the compound of formula Ia-H, the compound of formula Ia-I, and the compound of formula Ia-G.

4. Compound of formula (I) or (Ia) for use according to claim 1, administered orally, intraocularly, sublingually, intravenously, intramuscularly, intraarticularly, subcutaneously, transcutaneously, vaginally, peridurally, intravesically, rectally, or by inhalation.

5. Compound of formula (I) or (Ia) for use according to claim 1 in combination with at least one additional therapeutic agent.

6. Compound of formula (I) or (Ia) for use according to claim 5, wherein the at least one additional therapeutic agent is an analgesic, an NSAID, cortisone, a cortisone derivative, an immunosuppressant, an immunomodulator, an anti-TNF agent, an anti-interleukin agent, and combinations thereof.

7. Compound of formula (I) or (Ia) for use according to claim 6, wherein the at least one additional therapeutic agent is an immunosuppressant selected from methotrexate and cyclosporine, preferably methotrexate.

8. Composition comprising a compound of formula (I): 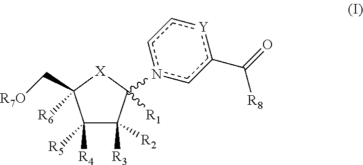
or a pharmaceutically acceptable stereoisomer, salt, hydrate, solvate, or crystal thereof, wherein
X is selected from O, CH₂, S, Se, CHF, CF₂, C═CH₂;
R₁ is selected from H, azido, cyano, C₁-C8 alkyl, C₁-C8 thioalkyl, C₁-C8 heteroalkyl, and OR;
wherein R is selected from H and C₁-C8 alkyl;
R₂, R₃, R₄, and R₅ are selected independently of one another from H, halogen, azido, cyano, hydroxyl, C₁-C12 alkyl, C₁-C12 thioalkyl, C₁-C12 heteroalkyl, C₁-C12 haloalkyl, and OR; wherein R is selected from H, C₁-C₁₂ alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)NH(C₁-C₁₂) alkylaryl, C(O)O(C₁-C₁₂) alkylaryl, and C(O)CHR_{AA}NH₂; wherein R_{AA} is a side chain selected from the proteinogenic amino acids;
R₆ is selected from H, azido, cyano, C₁-C8 alkyl, C₁-C8 thioalkyl, C₁-C8 heteroalkyl, and OR; wherein R is selected from H and C₁-C8 alkyl;
R₇ is selected from H, P(O)R₉R₁₀, and P(S)R₉R₁₀; wherein
R₉ and R₁₀ are selected independently of one another from OH, OR₁₁, NHR₁₃, NR₁₃R₁₄, C₁-C₈ alkyl, C₂-C₈ alkenyl, C₂-C₈ alkynyl, C₃-C10 cycloalkyl, C₅-C12 aryl, (C₁-C₈) alkylaryl, (C₁-C₈) arylalkyl, (C₁-C₈) heteroalkyl, (C₁-C₈) heterocycloalkyl, heteroaryl, and NHCHR_{A}R_{A′}C(O)R₁₂; wherein:
R₁₁ is selected from a C₁-C10 alkyl, C₃-C10 cycloalkyl, C₅-C18 aryl, C₁-C10 alkylaryl, substituted C₅-C12 aryl, C₁-C10 heteroalkyl, C₃-C10 heterocycloalkyl, C₁-C₁₀ haloalkyl, heteroaryl, —(CH₂)ₙC(O)(C₁-C₁₅) alkyl, —(CH₂)ₙOC(O)(C₁-C₁₅) alkyl, —(CH₂)ₙOC(O)O(C₁-C₁₅) alkyl, —(CH₂)ₙSC(O)(C₁-C₁₅) alkyl, —(CH₂)ₙC(O)O(C₁-C₁₅) alkyl, and —(CH₂)ₙC(O)O(C₁-C₁₅)alkylaryl group; wherein n is an integer from 1-8; P(O)(OH)OP(O)(OH)₂. halogen, nitro, cyano, C₁-C₆ alkoxy, C₁-C₆ haloalkoxy, —N(R₁₁ₐ)₂, C₁-C₆ acylamino, —COR_{11b}, —OCOR_{11b}; NHSO₂(C₁-C6 alkyl), —SO₂N(R₁₁ₐ)₂SO₂; wherein each R₁₁ₐ is independently selected from H and C₁-C₆ alkyl, and R_{11b} is independently selected from OH, C₁-C₆ alkoxy, NH₂, NH(C₁-C₆ alkyl), and N(C₁-C₆ alkyl)₂;
R₁₂ is selected from H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the aryl or heteroaryl groups are optionally substituted with one or two groups selected from halogen, trifluoromethyl, C₁-C₆ alkyl, C₁-C₆ alkoxy, and cyano; and
R_{A} and R_{A′} are independently selected from H, C₁-C10 alkyl, C₂-C10 alkenyl, C₂-C₁₀ alkynyl, C₃-C₁₀ cycloalkyl, C₁-C10 thioalkyl, C₁-C10 hydroxylalkyl, C₁-C10 alkylaryl, and C₅-C12 aryl, C₃-C10 heterocycloalkyl, heteroaryl, —(CH₂)₃NHC(═NH)NH₂, (1H-indol-3-yl) methyl, (1H-imidazol-4-yl) methyl, and a side chain selected from a proteinogenic or non-proteinogenic amino acid; wherein the aryl groups are optionally substituted with a group selected from hydroxyl, C₁-C10 alkyl, C₁-C6 alkoxy, halogen, nitro, and cyano; or
R₉ and R₁₀ form, together with the phosphorus atoms to which they are attached, a 6-membered cycle, wherein —R₉-R₁₀— is —CH₂—CH₂—CHR—; wherein R is selected from H, a (C₅-C₆) aryl, and a (C₅-C₆) heteroaryl group; wherein the aryl or heteroaryl groups are optionally substituted with halogen, trifluoromethyl, C₁-C₆ alkyl, C₁-C6 alkoxy, and cyano; or
R₉ and R₁₀ form, together with the phosphorus atoms to which they are attached, a 6-membered cycle, wherein —R₉-R₁₀— is —O—CH₂—CH₂—CHR—O—; wherein R is selected from H, a (C₅-C₆) aryl, and a (C₅-C₆) heteroaryl group; wherein the aryl or heteroaryl groups are optionally substituted with halogen, trifluoromethyl, (C₁-C₆) alkyl, (C₁-C6) alkoxy, and cyano;
R₈ is selected from H, OR, NHR₁₃, NR₁₃R₁₄, NH—NHR₁₃, SH, CN, N₃, and halogen; wherein R₁₃ and R₁₄ selected, independently of one another, from H, (C₁-C₈) alkyl, (C₁-C₈) alkylaryl, and —CR_{B}R_{C}—C(O)—OR_{D}; wherein R_{B} and R_{C} are independently a hydrogen atom, (C₁-C₆) alkyl, (C₁-C₆) alkoxy, benzyl, indolyl or imidazolyl; wherein the (C₁-C₆) alkyl and the (C₁-C₆) alkoxy may, optionally and independently of one another, be substituted by one or more halogen, amino, amido, guanidyl, hydroxyl, thiol, or carboxyl groups; and the benzyl group is optionally substituted by one or more halogen or hydroxyl groups; or R_{B} and R_{C}, together with the carbon atom to which they are attached, form a C₃-C₆ cycloalkyl group optionally substituted with one or more halogen, amino, amido, guanidyl, hydroxyl, thiol, and carboxyl; and R_{D} is hydrogen, (C₁-C₆) alkyl, (C₂-C₆) alkenyl, (C₂-C₆) alkynyl, or (C₃-C₆) cycloalkyl;
Y is selected from CH, CH₂, C(CH₃)₂, and CCH₃; [symbol]
is a single or double bond depending on Y; and [symbol]
is the alpha or beta anomer depending on the position of R₁
and/or
a compound of formula (Ia): 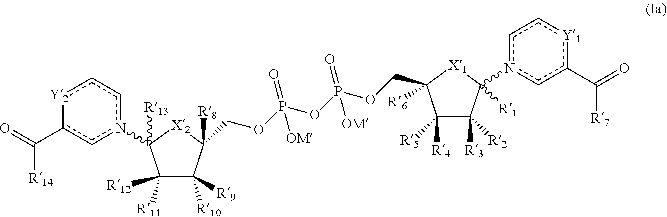
or a pharmaceutically acceptable stereoisomer, salt, hydrate, solvate, or crystal thereof, or a combination thereof, wherein
X′₁ and X′₂ are independently selected from O, CH₂, S, Se, CHF, CF₂, and C═CH₂;
R′₁ and R′13 are independently selected from H, azido, cyano, C1-C8 alkyl, C1-C8 thioalkyl, C1-C8 heteroalkyl, and OR; wherein R is selected from H and C₁-C8 alkyl;
R′₂, R′₃, R′₄, R′₅, R′₉, R′₁₀, R′₁₁, R′₁₂ are independently selected from H, halogen, azido, cyano, hydroxyl, C₁-C₁₂ alkyl, C₁-C₁₂ thioalkyl, C₁-C₁₂ heteroalkyl, C₁-C₁₂ haloalkyl, and OR; wherein R may be selected from H, C₁-C₁₂ alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)(C₁-C₁₂) aryl, C(O)NH(C₁-C₁₂) alkylaryl, C(O)O(C₁-C₁₂) alkylaryl, and a C(O)CHR_{AA}NH₂ group; wherein R_{AA} is a side chain selected from the proteinogenic amino acids;
R′₆ and R′₈ are independently selected from H, azido, cyano, C₁-C₈ alkyl, and OR, wherein R is selected from H and C₁-C₈ alkyl;
R′₇ and R′₁₄ are independently selected from H, OR, NHR, NRR′, NH—NHR, SH, CN, N₃, and halogen; wherein R and R′ are independently selected from H and (C₁-C₈) alkylaryl;
Y′1 and Y′2 are independently selected from CH, CH₂, C(CH₃)₂ and CCH₃;
M′ is selected from H and a suitable counterion; [symbol]
is a single or double bond, depending on Y′1 and Y2; and [symbol]
is an alpha or beta anomer depending on the position of R′₁ and R′₁₃;
and at least one pharmaceutically acceptable excipient for use in the prevention and/or treatment of ankylosing spondylitis.

9. Composition according to claim 8, further comprising at least one additional therapeutic agent.

10. Compound according to claim 9, wherein the at least one additional therapeutic agent is selected from an analgesic, an NSAID, cortisone, a cortisone derivative, an immunosuppressant, an immunomodulator, an anti-TNF agent, an anti-interleukin agent, and combinations thereof.

11. Composition according to claim 8, wherein it is administered orally, intraocularly, sublingually, intravenously, intraarterially, intramuscularly, intraarticularly, subcutaneously, transcutaneously, vaginally, peridurally, intravesically, rectally, or by inhalation

12. Composition according to claim 11, wherein it may be administered in the form of a sublingual tablet or a gastroresistant capsule.

13. Composition according to claim 8, wherein the compound of formula (I) is selected from compound I-A, compound I-B, compound I-C, compound I-D, compound I-E, compound I-F, compound I-G, compound I-H, compound I-I, compound I-J, preferably compound I-C, compound I-D, or compound I-F.

14. Composition according to claim 8, wherein the compound of formula (Ia) is selected from the compound of formula Ia-B, the compound of, wherein the compound of formula I is selected from the compound of formula Ia-C, the compound of formula Ia-E, the compound of formula Ia-F, the compound of formula Ia-H, the compound of formula Ia-I, and the compound of formula Ia-G, and combinations thereof.

15. Combination preparation comprising a compound of formula (I) and/or a compound of formula (Ia) according to claim 1, and/or a composition comprising the compound of formula (I) and/or the compound of formula (Ia) and at least one additional therapeutic agent for use in the prevention and/or treatment of ankylosing spondylitis.
